(19) United States
(12) Patent Application Publication
(10) Pub. No.: US 2025/0136550 A1
(43) Pub. Date: May 1, 2025

(54) PROCESSES FOR PREPARING REVEFENACIN AND COMPOSITIONS COMPRISING THE SAME

(71) Applicant: THERAVANCE BIOPHARMA R&D IP, LLC, South San Francisco, CA (US)

(72) Inventors: James Wertman, San Mateo, CA (US); Kenley Ngim, Walnut Creek, CA (US); Bradley Kumagai, Palo Alto, CA (US); Bridget May, San Jose, CA (US)

(21) Appl. No.: 18/496,404

(22) Filed: Oct. 27, 2023

(51) Int. Cl. C07D 211/62

(52) U.S. Cl. CPC C07D 211/62; C07B 2200/13

(57) ABSTRACT

The present disclosure relates to intermediates and processes for preparing biphenyl-2-ylcarbamic acid 1-(2-{[4-(4-carbamoylpiperidin-1-ylmethyl) benzoyl]methylamino}ethyl)piperidin-4-yl ester and solid forms thereof, and compositions comprising the same.

### FIELD

The present disclosure relates to intermediates and processes for preparing biphenyl-2-ylcarbamic acid 1-(2-{[4-(4-carbamoylpiperidin-1-ylmethyl) benzoyl]methylamino}ethyl)piperidin-4-yl ester and solid forms thereof, and compositions comprising the same.

### BACKGROUND

Revefenacin, or biphenyl-2-ylcarbamic acid 1-(2-{[4-(4-carbamoylpiperidin-1-ylmethyl)benzoyl]methylamino}-ethyl)piperidin-4-yl ester, is a medication for the treatment of chronic obstructive pulmonary disease (COPD). Revefenacin was approved for use in the United States in 2018.

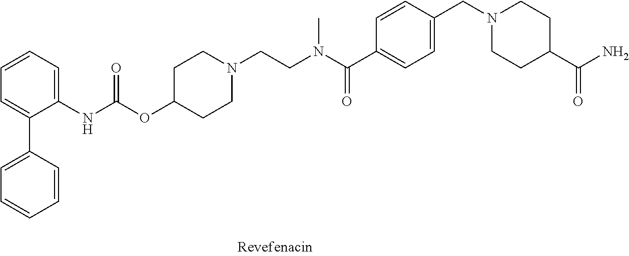 [SMILES: CN(CCN1CCC(OC(=O)Nc2ccccc2-c2ccccc2)CC1)C(=O)c1ccc(CN2CCC(C(N)=O)CC2)cc1]

Thus, a need exists for an efficient process of preparing revefenacin as a pure material with high chemical purity and good overall yield, without having to isolate intermediates.

### SUMMARY

Provided herein are processes for providing revefenacin, wherein the revefenacin may be amorphous or crystalline (e.g., crystalline revefenacin Form I, crystalline revefenacin Form II, crystalline revefenacin Form III, crystalline revefenacin Form IV, or amorphous revefenacin), as well as intermediates therefor, and processes for providing the same in high purity.

In one aspect, provided is a composition comprising Compound 3:

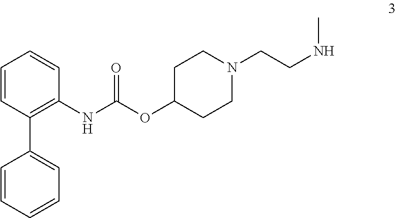 [SMILES: CNCCN1CCC(OC(=O)Nc2ccccc2-c2ccccc2)CC1]

wherein the composition is substantially free of certain impurities and/or residual starting materials, as well as processes and compounds for preparing the same.

Also provided is a composition comprising Compound 4:

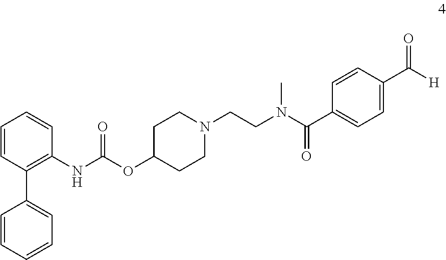 [SMILES: CN(CCN1CCC(OC(=O)Nc2ccccc2-c2ccccc2)CC1)C(=O)c1ccc(C=O)cc1]

wherein the composition is substantially free of certain impurities and/or residual starting materials, as well as processes and compounds for preparing the same.

In certain aspects, provided is a process for preparing Compound 2, Compound 3, Compound 4, or revefenacin (including, but not limited to, crystalline revefenacin Form I, crystalline revefenacin Form II, crystalline revefenacin Form III, crystalline revefenacin Form IV, amorphous revefenacin, or salts of revefenacin including crystalline salts), said process comprising adding a composition comprising a quantity of Compound 1 to a composition comprising Compound A:

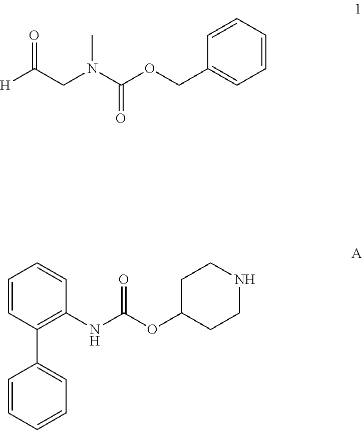 [SMILES: CN(CC=O)C(=O)OCc1ccccc1 ; O=C(Nc1ccccc1-c1ccccc1)OC1CCNCC1]

under conditions sufficient to provide a composition comprising Compound 2:

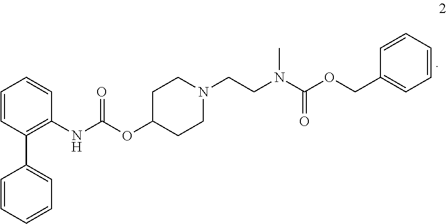 [SMILES: CN(CCN1CCC(OC(=O)Nc2ccccc2-c2ccccc2)CC1)C(=O)OCc1ccccc1]

### DETAILED DESCRIPTION

The presents disclosure relates to processes for providing revefenacin, wherein the revefenacin may be amorphous or crystalline (e.g., crystalline revefenacin Form I, crystalline revefenacin Form II, crystalline revefenacin Form III, crystalline revefenacin Form IV, amorphous revefenacin, or salts thereof), as well as intermediates therefor, and processes for providing the same in high purity.

### Definitions

The following terms have the following meanings unless otherwise indicated. Additionally, as used herein, the singular forms “a,” “an,” and “the” include the corresponding plural forms unless the context of use clearly dictates otherwise. The terms “comprising,” “including,” and “having” are intended to be inclusive and mean that there may be additional elements other than the listed elements. All numbers expressing quantities of ingredients, properties such as molecular weight, reaction conditions, and so forth used herein are to be understood as being modified in all instances by the term “about,” unless otherwise indicated. Accordingly, the numbers set forth herein are approximations that may vary depending upon the desired properties sought to be obtained by the present disclosure. At least, and not as an attempt to limit the application of the doctrine of equivalents to the scope of the claims, each number should at least be construed in light of the reported significant digits and by applying ordinary rounding techniques.

As used herein, the phrase “having the formula” or “having the structure” is not intended to be limiting and is used in the same way that the term “comprising” is commonly used. All other terms used herein are intended to have their ordinary meaning as understood by those of ordinary skill in the art to which they pertain.

### Processes

It will be appreciated that while specific process conditions (i.e. temperatures, times, mole ratios of reactants, solvents, pressures, etc.) are given, other process conditions can also be used unless otherwise stated. The molar ratios described in the methods of the disclosure can be readily determined by various methods available to those skilled in the art. For example, such molar ratios can be readily determined by ¹H NMR. Alternatively, elemental analysis and HPLC methods can be used to determine the molar ratio. Further, as is well known in the field of powder x-ray diffraction, relative peak heights of powder x-ray diffraction (PXRD) spectra are dependent on a number of factors relating to sample preparation and instrument geometry, while peak positions are relatively insensitive to experimental details.

Provided herein are processes for providing revefenacin, wherein the revefenacin may be amorphous or crystalline (e.g., crystalline revefenacin Form I, crystalline revefenacin Form II, crystalline revefenacin Form III, crystalline revefenacin Form IV, or amorphous revefenacin), as well as intermediates therefor and processes for providing the same in high purity.

Processes for providing revefenacin have been reported (see, e.g., U.S. Pat. No. 8,754,225). Scheme I, below, shows one overall process for providing revefenacin.

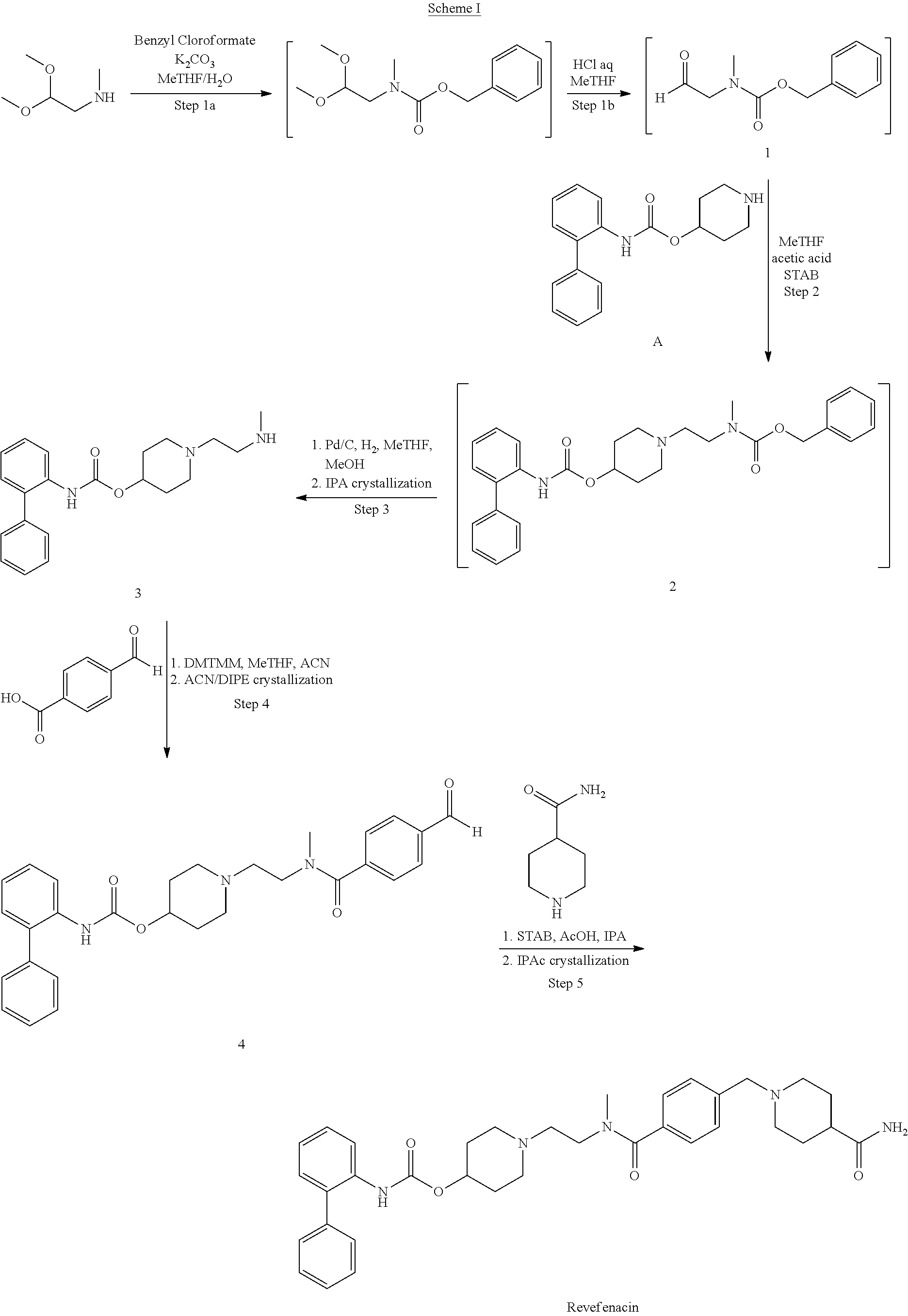

In some embodiments, the processes described herein provide revefenacin, as well as certain intermediates, in high purity.

### Compound 3

As shown in Scheme I, Compound 3 is prepared by contacting Compound 1 with Compound A under conditions to provide Compound 2, followed by hydrogenating Compound 2.

In some embodiments, provided herein is a process for providing Compound 3 (e.g., a composition comprising Compound 3) in high purity. In some embodiments, the amount of certain side products formed in the reaction to provide Compound 3 are diminished (e.g., Compound 1A and Compound 1B).

Accordingly, in some embodiments, provided is a composition comprising Compound 3:

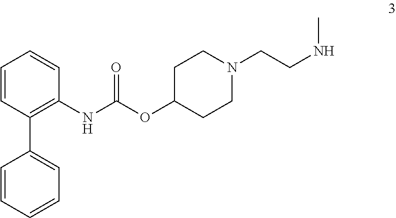 [SMILES: CNCCN1CCC(OC(=O)Nc2ccccc2-c2ccccc2)CC1]

wherein the composition comprises less than 0.3% Compound 1A:

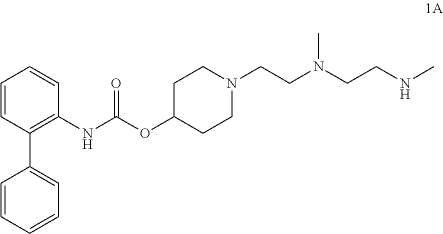 [SMILES: CNCCN(C)CCN1CCC(OC(=O)Nc2ccccc2-c2ccccc2)CC1]

or less than 0.55% Compound 1B:

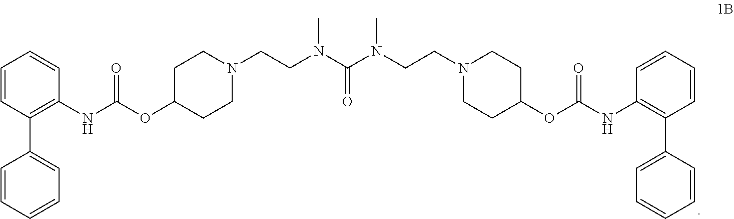 [SMILES: CN(CCN1CCC(OC(=O)Nc2ccccc2-c2ccccc2)CC1)C(=O)N(C)CCN1CCC(OC(=O)Nc2ccccc2-c2ccccc2)CC1]

In some embodiments, a process described herein provides Compound 3 (e.g., a composition comprising Compound 3) wherein the amount of starting material in the final composition is diminished. In some embodiments, the composition comprises less than 0.67% Compound A:

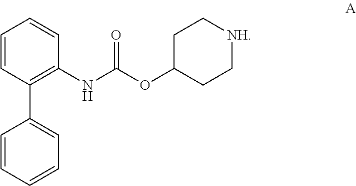 [SMILES: O=C(Nc1ccccc1-c1ccccc1)OC1CCNCC1]

In some embodiments, provided is a process for preparing a composition comprising Compound 3, wherein the composition comprises less than 0.3% Compound 1A or less than 0.55% Compound 1B, the process comprising:
a) combining a composition comprising a quantity of Compound 1 with a composition comprising Compound A, such that the quantity of Compound 1 added is less than 75% by weight, or less than 73% by weight, or from 67% to 75% by weight, or from 70% to 72% by weight, or 71% by weight, the quantity of Compound A:

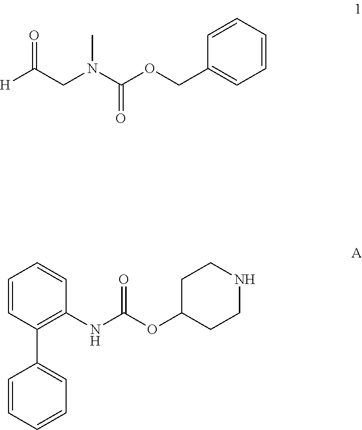 [SMILES: CN(CC=O)C(=O)OCc1ccccc1 ; O=C(Nc1ccccc1-c1ccccc1)OC1CCNCC1]

under conditions sufficient to provide a composition comprising Compound 2:

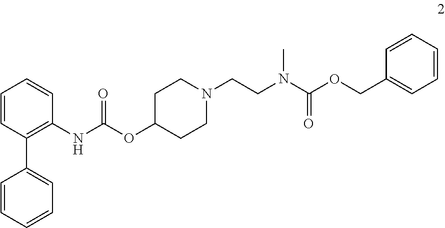 [SMILES: CN(CCN1CCC(OC(=O)Nc2ccccc2-c2ccccc2)CC1)C(=O)OCc1ccccc1]

and
b) hydrogenating the composition comprising Compound 2 under conditions sufficient to provide the composition comprising Compound 3.

In some embodiments, provided is a process for preparing a composition comprising Compound 3, wherein the composition comprises less than 0.3% Compound 1A or less than 0.55% Compound 1B, the process comprising:
a) adding a composition comprising a quantity of Compound 1 to a composition comprising Compound A, such that the quantity of Compound 1 added is less than 75% by weight, or less than 73% by weight, or from 67% to 75% by weight, or from 70% to 72% by weight, or 71% by weight, the quantity of Compound A:

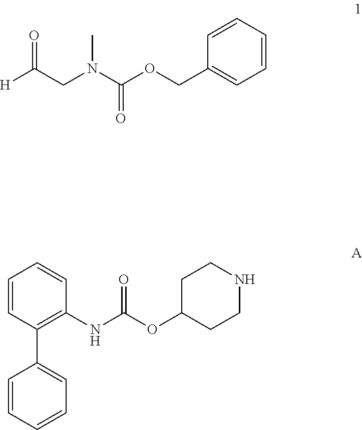 [SMILES: CN(CC=O)C(=O)OCc1ccccc1 ; O=C(Nc1ccccc1-c1ccccc1)OC1CCNCC1]

under conditions sufficient to provide a composition comprising Compound 2:

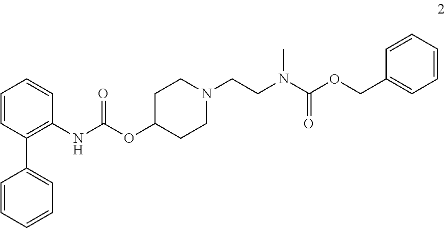 [SMILES: CN(CCN1CCC(OC(=O)Nc2ccccc2-c2ccccc2)CC1)C(=O)OCc1ccccc1]

and b) hydrogenating the composition comprising Compound 2 under conditions sufficient to provide the composition comprising Compound 3.

In some embodiments, Compound 3 is isolated. In some embodiments, Compound 3 is isolated via crystallization. In some embodiments, Compound 3 is isolated via crystallization from isopropyl ether.

In some embodiments, the quantity of Compound 1 added is less than 75% by weight, or less than 74% by weight, or less than 73% by weight, or less than 72% by weight, or less than 71% by weight, or less than 70% by weight, or less than 69% by weight, or less than 68% by weight, or less than 67% by weight, between 67-75% by weight, between 67-74% by weight, between 67-73% by weight, between 67-72% by weight, between 67-71% by weight, between 67-70% by weight, between 70-74% by weight, between 70-73% by weight, between 70-72% by weight, the quantity of Compound A. In some embodiments, the quantity of Compound 1 added is between 67-72% by weight the quantity of Compound A. The quantity of Compound 1 added in step a) can be determined, monitored, or controlled by first determining the concentration of Compound 1 in a composition.

In some embodiments, the process conditions comprise a solvent system comprising 2-methyltetrahydrofuran (MeTHF). In some embodiments, the process comprises acetic acid. In some embodiments, the process comprises about 20% by weight acetic acid, or from about 10-30% by weight acetic acid. In some embodiments, the process conditions comprise a solvent system comprising acetic acid and 2-methyltetrahydrofuran (MeTHF).

In some embodiments, the process conditions of step a) comprise a reducing agent. In some embodiments, the reducing agent is a borohydride reducing agent. In some embodiments, the reducing agent is sodium triacetoxyhydroborate.

In some embodiments, the process conditions of step a) comprise a temperature of below about 15° C., or below about 10° C., or about −5 to 15° C., or about 5 to 15° C., or about 5 to 10° C., or about 8° C. In some embodiments, the process conditions of step a) comprise an addition time of from about 0.1-2 hours, or from about 0.5 to 1.5 hours, or about 1 hour.

Once the quantity is determined, an appropriate amount of Compound 1 is reacted with Compound A to provide compound 2. Hydrogenating the composition comprising Compound 2 under conditions sufficient provides a composition comprising Compound 3.

In some embodiments, the hydrogenating (step b)) comprises a palladium catalyst. In some embodiments, the hydrogenating comprises hydrogen gas and palladium catalyst. In some embodiments, the hydrogenating comprises a solvent system comprising methanol and MeTHF. In some embodiments, the organic solvent comprises MeTHF and acetic acid. In some embodiments, the hydrogenating (step b)) comprises a temperature of from about 20 to 40° C., or from about 25-35° C., or about 30° C.

As shown in Scheme I, Compound 1 is formed in solution and combined with Compound A without being isolated. As such, in order to react a predetermined quantity of Compound A with Compound 1 and decrease impurity and residual starting materials in Compound 3, a method for determining the quantity of Compound 1 in a solution is required.

In some embodiments, disclosed herein is a process for determining the quantity of Compound 1 comprising reacting an aliquot of the composition comprising Compound 1 with an appropriate modifying group, such that the product formed thereby can be analyzed by appropriate methods, such as, but not limited to HPLC.

In some embodiments, the step of determining the quantity of Compound 1 in a solution comprises labeling Compound 1 with a tag to provide a derivative visible by HPLC. In some embodiments, the labeling comprises reacting the aldehyde functional group in Compound 1 with a hydrazine compound, or hydrazine derivative, such as a hydrazide, semicarbazide, carbohydrazide, or hydroxylamine containing compound. Hydrazine derivatives react with aldehydes to yield hydrazones. Hydroxylamine derivatives (aminooxy compounds) react with aldehydes to yield oximes. Both hydrazones and oximes can be reduced (e.g., with NaBH₄) to further increase the stability of the linkage. Rates and yields of aldehyde reactions with hydrazine and hydroxylamine derivatives can be enhanced by aniline catalysis.

The resulting labeled Compound 1 can be excited with UV or visible light, depending on the desired detection method.

In some embodiments, the step of determining comprises reacting an aliquot of the composition comprising Compound 1 with a hydrazine (Compound t) to provide the corresponding hydrazone (Compound 1-t):

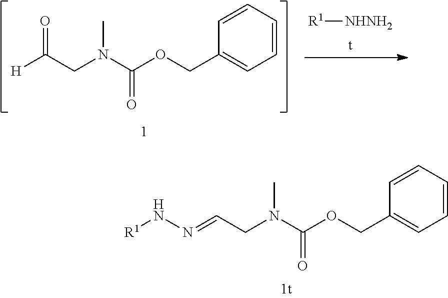

wherein the quantity of Compound 1-t is determined by HPLC.

The quantity of hydrazone can be determined by standard methods, including plotting a calibration curve using a standard marker (either isolated and identified or procured from suitable source). As such, the quantity of Compound 1 can be determined in solution.

In some embodiments, the hydrazine (Compound t) is 2,4-dinitrophenylhydrazine (2,4-DNPH or DNPH), 4-dimethylamino-6-(4-methoxy-1-naphthyl)-1,3,5-triazine-2-hydrazine (2-hydrazino-4-(dimethylamino)-6-(4-methoxy-1-naphthyl)-1,3,5-triazine or DMNTH), 4-hydrazinobenzoic acid, or 3-nitrophenylhydrazine (3NPH).

In some embodiments, R¹ is:

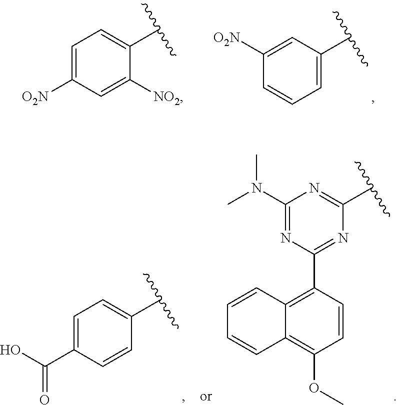 [SMILES: *c1ccc([N+](=O)[O-])cc1[N+](=O)[O-] ; *c1cccc([N+](=O)[O-])c1 ; *c1ccc(C(=O)O)cc1 ; *c1nc(-c2ccc(OC)c3ccccc23)nc(N(C)C)n1]

In some embodiments, R¹ is R¹ is 2,4-dinitrophenyl.

In some embodiments, the step of determining the quantity of Compound 1 comprises reacting an aliquot of a composition comprising Compound 1 with 2,4-dinitrophenylhydrazine to provide benzyl (E)-(2-(2-(2,4-dinitrophenyl)hydrazineylidene)ethyl)(methyl)carbamate (Compound 10):

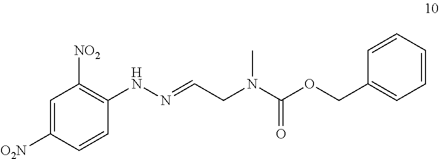 [SMILES: CN(C/C=N/Nc1ccc([N+](=O)[O-])cc1[N+](=O)[O-])C(=O)OCc1ccccc1]

and determining the quantity of benzyl (E)-(2-(2-(2,4-dinitrophenyl)hydrazineylidene)ethyl)(methyl)carbamate (Compound 10) by HPLC.

In some embodiments, the step of determining the quantity of Compound 1 comprises reacting an aliquot of a composition comprising Compound 1 with 2,4-dinitrophenylhydrazine (2,4-DNPH or DNPH), 4-dimethylamino-6-(4-methoxy-1-naphthyl)-1,3,5-triazine-2-hydrazine (2-hydrazino-4-(dimethylamino)-6-(4-methoxy-1-naphthyl)-1,3,5-triazine or DMNTH), 4-hydrazinobenzoic acid, or 3-nitrophenylhydrazine (3NPH) to provide the corresponding hydrazone thereof, and determining the quantity of hydrazone by HPLC.

In one embodiment, provided is a compound (e.g., a hydrazone) having the structure:

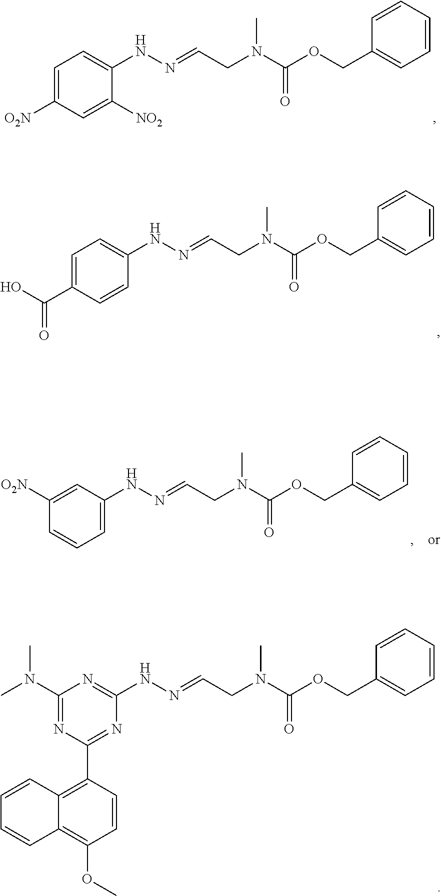 [SMILES: CN(C/C=N/Nc1ccc([N+](=O)[O-])cc1[N+](=O)[O-])C(=O)OCc1ccccc1 ; CN(C/C=N/Nc1ccc(C(=O)O)cc1)C(=O)OCc1ccccc1 ; CN(C/C=N/Nc1cccc([N+](=O)[O-])c1)C(=O)OCc1ccccc1 ; COc1ccc(-c2nc(N/N=C/CN(C)C(=O)OCc3ccccc3)nc(N(C)C)n2)c2ccccc12]

In one embodiment, provided herein is benzyl (E)-(2-(2-(2,4-dinitrophenyl)hydrazineylidene) ethyl)(methyl)carbamate (Compound 10):

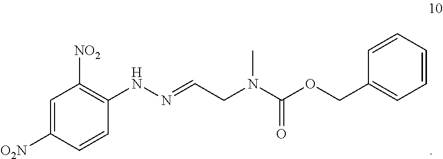 [SMILES: CN(C/C=N/Nc1ccc([N+](=O)[O-])cc1[N+](=O)[O-])C(=O)OCc1ccccc1]

In one embodiment, provided herein is a composition comprising benzyl (E)-(2-(2-(2,4-dinitrophenyl)hydrazineylidene)ethyl)(methyl)carbamate (Compound 10):

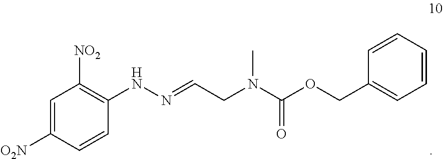 [SMILES: CN(C/C=N/Nc1ccc([N+](=O)[O-])cc1[N+](=O)[O-])C(=O)OCc1ccccc1]

In one embodiment, provided herein is a process for providing a composition comprising benzyl (E)-(2-(2-(2,4-dinitrophenyl)hydrazineylidene)ethyl)(methyl)carbamate (Compound 10):

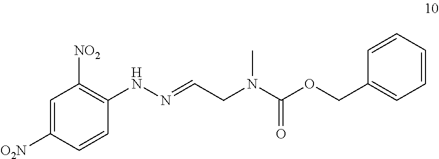 [SMILES: CN(C/C=N/Nc1ccc([N+](=O)[O-])cc1[N+](=O)[O-])C(=O)OCc1ccccc1]

comprising contacting Compound 1 with 2,4-dinitrophenylhydrazine under conditions sufficient to provide the composition comprising benzyl (E)-(2-(2-(2,4-dinitrophenyl)hydrazineylidene)ethyl)(methyl)carbamate (Compound 10).

In some embodiments, the contacting comprises a solvent. In one embodiment, the solvent is MeTHF.

In one embodiment, an excess of 2,4-dinitrophenylhydrazine is added to Compound 1 (e.g., a solution of Compound 1 in a solvent, such as MeTHF). In one embodiment, a 1.5-3 fold molar excess (i.e., mol/mol) of the 2,4-dinitrophenylhydrazine is added to Compound 1 (e.g., a solution of Compound 1 in a solvent, such as MeTHF).

### Compound 4

As shown in Scheme I, Compound 4 is prepared by contacting Compound 3 with 4-formylbenzoic acid under conditions to provide Compound 4.

In some embodiments, provided herein is a process for providing Compound 4 (e.g., a composition comprising Compound 4) in high purity. In some embodiments, the amount of certain side products present or formed in the reaction providing Compound 4 are diminished (e.g., Compound 3A and Compound 1B).

Accordingly, in some embodiments, provided is a composition comprising Compound 4:

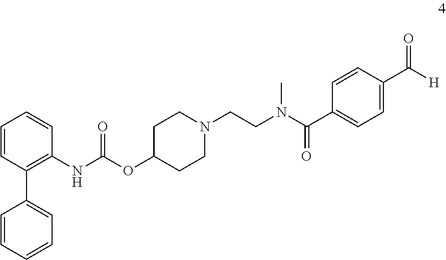 [SMILES: CN(CCN1CCC(OC(=O)Nc2ccccc2-c2ccccc2)CC1)C(=O)c1ccc(C=O)cc1]

wherein the composition comprises less than 0.55% Compound 3A:

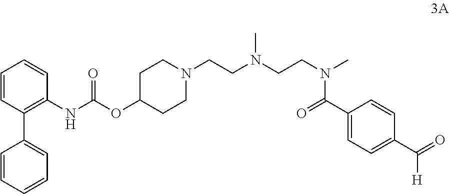 [SMILES: CN(CCN1CCC(OC(=O)Nc2ccccc2-c2ccccc2)CC1)CCN(C)C(=O)c1ccc(C=O)cc1]

or less than 0.55% Compound 1B:

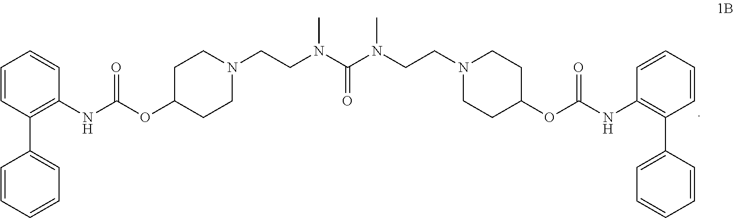 [SMILES: CN(CCN1CCC(OC(=O)Nc2ccccc2-c2ccccc2)CC1)C(=O)N(C)CCN1CCC(OC(=O)Nc2ccccc2-c2ccccc2)CC1]

In some embodiments, provided is a process for providing the composition comprising Compound 4, said process comprising contacting the composition comprising Compound 3 as disclosed herein, with 4-formylbenzoic acid under conditions sufficient to provide a composition comprising Compound 4. In some embodiments, the process for providing a composition of Compound 4 comprises an organic triazine, such as DMTMM (4-(4,6-dimethoxy-1,3,5-triazin-2-yl)-4-methyl-morpholinium chloride).

In some embodiments, the process for providing a composition of Compound 4 comprises contacting a composition comprising Compound 3 as disclosed herein, with 4-formylbenzoic acid under conditions sufficient to provide a composition of Compound 4 with less than 0.55%, or less than 0.50%, or less than 0.45%, or less than 0.40%, or less than 0.35%, or less than 0.30%, or less than 0.25%, or less than 0.20%, or less than 0.15%, or less than 0.10%, or less than 0.06% a/a (relative area on HPLC) Compound 3A.

In some embodiments, the process for providing a composition of Compound 4 comprises contacting a composition comprising Compound 3 as disclosed herein, with 4-formylbenzoic acid under conditions sufficient to provide a composition of Compound 4 with less than 0.55%, or less than 0.50%, or less than 0.45%, or less than 0.40%, or less than 0.35%, or less than 0.30%, or less than 0.25%, or less than 0.20%, or less than 0.15%, or less than 0.10%, or less than 0.05% a/a (relative area on HPLC) Compound 1B.

In some embodiments, the process for providing the composition of Compound 4 comprises acetonitrile and optionally MeTHF as a reaction solvent. In some embodiments, the process for providing the composition of Compound 4 occurs at a temperature of from about 10 to 30° C., or from about 15-20° C., or about 17° C.

In some embodiments, Compound 4 is isolated. In some embodiments, Compound 4 is isolated via crystallization. In some embodiments, Compound 4 is isolated via crystallization from acetonitrile and diisopropylether (DIPE).

### Revefenacin

Provided herein are processes for providing revefenacin. The revefenacin as provided by the processes and intermediates and compositions disclosed herein, may be amorphous or crystallized to provide crystalline revefenacin (e.g., crystalline revefenacin Form I, crystalline revefenacin Form II, crystalline revefenacin Form III, or crystalline revefenacin Form IV).

As referred to herein, “crystalline revefenacin Form I” and “crystalline revefenacin Form II” refer to Form I and Form II of the revefenacin crystalline freebase as described in WO2006099165A1. As such, characterization and methods for preparing crystalline revefenacin Form I and crystalline revefenacin Form II are known in the art.

In some embodiments, provided is a process for preparing crystalline revefenacin Form I, crystalline revefenacin Form II, or amorphous revefenacin, said process comprising:
i) contacting a composition comprising Compound 3 as disclosed herein with 4-formylbenzoic acid under conditions sufficient to provide Compound 4:

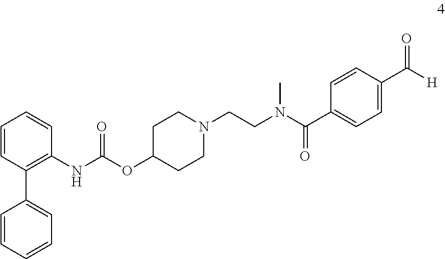 [SMILES: CN(CCN1CCC(OC(=O)Nc2ccccc2-c2ccccc2)CC1)C(=O)c1ccc(C=O)cc1]

ii) contacting Compound 4 with piperidine-4-carboxamide under conditions sufficient to provide revefenacin:

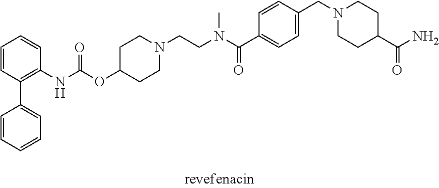 [SMILES: CN(CCN1CCC(OC(=O)Nc2ccccc2-c2ccccc2)CC1)C(=O)c1ccc(CN2CCC(C(N)=O)CC2)cc1]

and optionally:
iii) crystallizing the revefenacin under conditions sufficient to provide crystalline revefenacin Form I or crystalline revefenacin Form II.

In some embodiments, the process comprises crystallizing the revefenacin under conditions sufficient to provide crystalline revefenacin Form I. In some embodiments, the process comprises water as part of a solvent mixture as the inert diluent. In one embodiment, the crystalline revefenacin Form I is characterized by a PXRD pattern having one, two or more diffraction peaks at °2θ values selected from 4.7±0.2, 9.6±0.2, 12.7±0.2, 13.7±0.2, 16.7±0.2, 17.4±0.2, 18.5±0.2, 19.4±0.2, 20.8±0.2, 21.4±0.2, 24.2±0.2, and 25.6±0.2. In one embodiment, this crystalline form is characterized by a PXRD pattern comprising diffraction peaks at °2θ values of 4.7±0.2, 18.5±0.2, 20.8±0.2, and 25.6±0.2.

In some embodiments, the process comprises crystallizing the revefenacin under conditions sufficient to provide crystalline revefenacin Form II. In some embodiments, the process comprises an organic solvent mixture as the inert diluent, i.e., no water. In one embodiment, the crystalline revefenacin Form II is characterized by a PXRD pattern having one, two or more diffraction peaks at °2θ values selected from 4.6±0.2, 9.3±0.2, 12.9±0.2, 13.6±0.2, 14.0±0.2, 14.6±0.2, 16.5±0.2, 18.6±0.2, 19.1±0.2, 20.9±0.2, 22.1±0.2, 22.7±0.2, and 25.7±0.2. In one particular embodiment, this crystalline form is characterized by a PXRD pattern comprising diffraction peaks at °2θ values of 4.6±0.2, 18.6±0.2, 22.1±0.2, and 22.7±0.2.

Further characterizing data for crystalline revefenacin Form I or crystalline revefenacin Form II can be found in WO2006099165A1, which disclosure is incorporated by reference in its entirety.

In some embodiments, provided is a process for preparing crystalline revefenacin Form I, crystalline revefenacin Form II, or amorphous revefenacin, said process comprising:
i) contacting the composition of a composition comprising Compound 4 as described herein, with piperidine-4-carboxamide, under conditions sufficient to provide revefenacin:

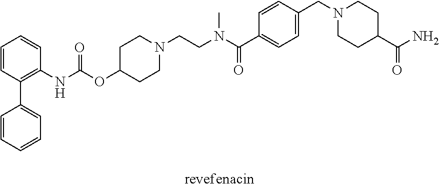 [SMILES: CN(CCN1CCC(OC(=O)Nc2ccccc2-c2ccccc2)CC1)C(=O)c1ccc(CN2CCC(C(N)=O)CC2)cc1]

and optionally:
ii) crystallizing the revefenacin under conditions sufficient to provide the crystalline revefenacin Form I or crystalline revefenacin Form II.

In some embodiments, provided is a process for preparing crystalline revefenacin Form I, crystalline revefenacin Form II, or amorphous revefenacin, said process comprising:
a) adding a composition comprising Compound 1 to a composition comprising Compound A:

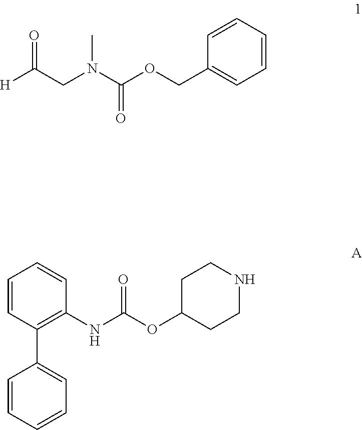 [SMILES: CN(CC=O)C(=O)OCc1ccccc1 ; O=C(Nc1ccccc1-c1ccccc1)OC1CCNCC1]

under conditions sufficient to provide a composition comprising Compound 2:

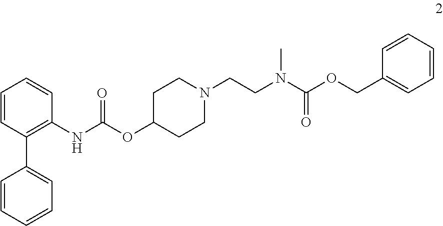 [SMILES: CN(CCN1CCC(OC(=O)Nc2ccccc2-c2ccccc2)CC1)C(=O)OCc1ccccc1]

wherein the quantity of Compound 1 added is determined by reacting an aliquot of the composition comprising Compound 1 with 2,4-dinitrophenylhydrazine under conditions sufficient to provide benzyl (E)-(2-(2-(2,4-dinitrophenyl)hydrazineylidene)ethyl)(methyl)carbamate (Compound 10):

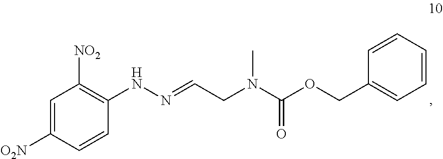 [SMILES: CN(C/C=N/Nc1ccc([N+](=O)[O-])cc1[N+](=O)[O-])C(=O)OCc1ccccc1]

and determining the quantity of benzyl (E)-(2-(2-(2,4-dinitrophenyl)hydrazineylidene)ethyl)(methyl)carbamate by HPLC;
b) hydrogenating the composition comprising Compound 2 under conditions sufficient to provide Compound 3:

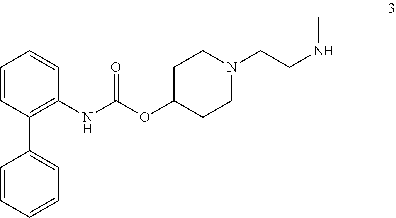 [SMILES: CNCCN1CCC(OC(=O)Nc2ccccc2-c2ccccc2)CC1]

c) contacting Compound 3 with 4-formylbenzoic acid under conditions sufficient to provide

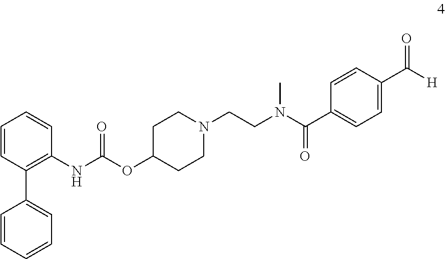 [SMILES: CN(CCN1CCC(OC(=O)Nc2ccccc2-c2ccccc2)CC1)C(=O)c1ccc(C=O)cc1]

d) contacting Compound 4 with piperidine-4-carboxamide under conditions sufficient to provide revefenacin:

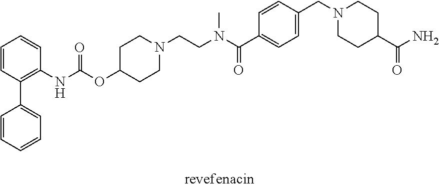 [SMILES: CN(CCN1CCC(OC(=O)Nc2ccccc2-c2ccccc2)CC1)C(=O)c1ccc(CN2CCC(C(N)=O)CC2)cc1]

and optionally:
e) crystallizing the revefenacin under conditions sufficient to provide the crystalline revefenacin Form I or crystalline revefenacin Form II.

In some embodiments, the conditions of step d) comprise a reducing agent, such as a borohydride reducing agent (e.g., sodium triacetoxyborohydride). In some embodiments, the conditions of step d) comprise an organic acid (e.g., acetic acid). In some embodiments, the conditions of step d) comprise a solvent (e.g., IPA).

In some embodiments, provided is a process for preparing crystalline revefenacin Form I, crystalline revefenacin Form II, or amorphous revefenacin, said process comprising:
a) contacting a composition comprising Compound 3:

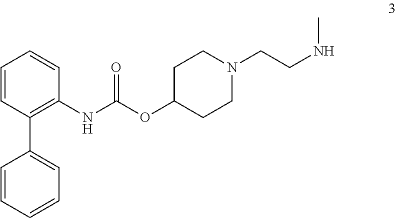 [SMILES: CNCCN1CCC(OC(=O)Nc2ccccc2-c2ccccc2)CC1]

wherein the composition comprises less than 0.3% Compound 1A:

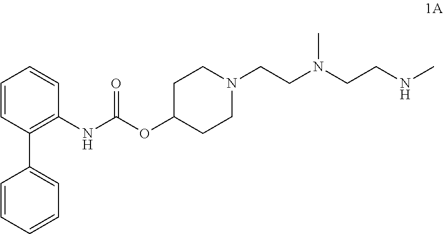 [SMILES: CNCCN(C)CCN1CCC(OC(=O)Nc2ccccc2-c2ccccc2)CC1]

or less than 0.55% Compound 1B:

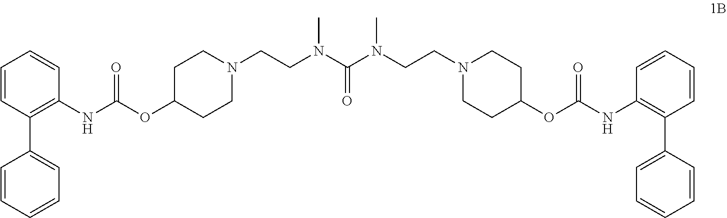 [SMILES: CN(CCN1CCC(OC(=O)Nc2ccccc2-c2ccccc2)CC1)C(=O)N(C)CCN1CCC(OC(=O)Nc2ccccc2-c2ccccc2)CC1]

or less than 0.67% Compound A:

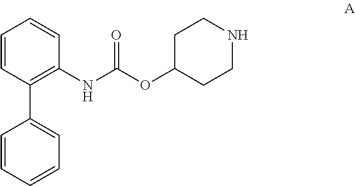 [SMILES: O=C(Nc1ccccc1-c1ccccc1)OC1CCNCC1]

with 4-formylbenzoic acid to provide Compound 4:

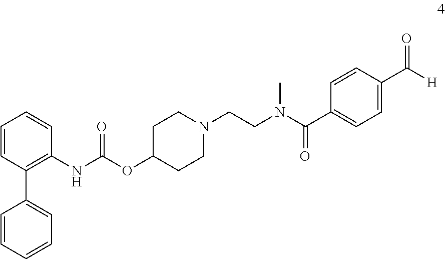 [SMILES: CN(CCN1CCC(OC(=O)Nc2ccccc2-c2ccccc2)CC1)C(=O)c1ccc(C=O)cc1]

b) contacting Compound 4 with piperidine-4-carboxamide under conditions sufficient to provide revefenacin:

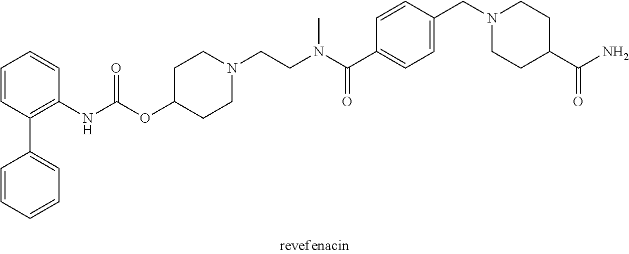 [SMILES: CN(CCN1CCC(OC(=O)Nc2ccccc2-c2ccccc2)CC1)C(=O)c1ccc(CN2CCC(C(N)=O)CC2)cc1]

and optionally:
c) crystallizing the revefenacin under conditions sufficient to provide the crystalline revefenacin Form I or crystalline revefenacin Form II.

In some embodiments, provided is a process for preparing crystalline revefenacin Form I, crystalline revefenacin Form II, or amorphous revefenacin, said process comprising:
a) adding a composition comprising Compound 1 to a composition comprising Compound A, such that the quantity of Compound 1 added is less than 75% by weight, or less than 74% by weight, or less than 73% by weight, or less than 72% by weight, or less than 71% by weight, or less than 70% by weight, or less than 69% by weight, or less than 68% by weight, or less than 67% by weight, between 67-75% by weight, between 67-74% by weight, between 67-73% by weight, between 67-72% by weight, between 67-71% by weight, between 67-70% by weight, between 70-74% by weight, between 70-73% by weight, between 70-72% by weight, the quantity of Compound A:

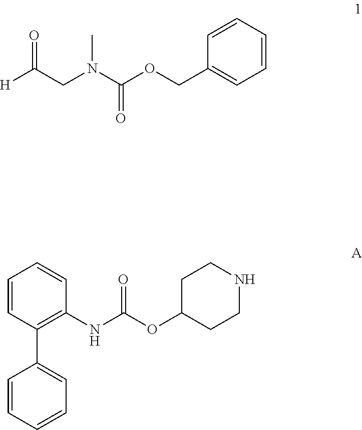 [SMILES: CN(CC=O)C(=O)OCc1ccccc1 ; O=C(Nc1ccccc1-c1ccccc1)OC1CCNCC1]

under conditions sufficient to provide Compound 2:

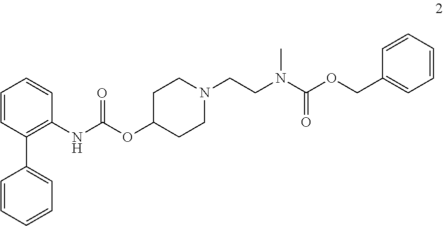 [SMILES: CN(CCN1CCC(OC(=O)Nc2ccccc2-c2ccccc2)CC1)C(=O)OCc1ccccc1]

b) hydrogenating the composition comprising Compound 2 under conditions to provide Compound 3:

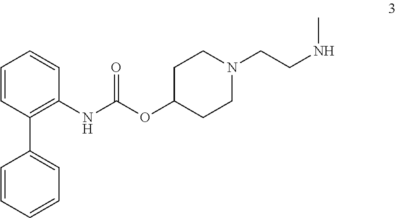 [SMILES: CNCCN1CCC(OC(=O)Nc2ccccc2-c2ccccc2)CC1]

c) contacting Compound 3 with 4-formylbenzoic acid, to provide Compound 4:

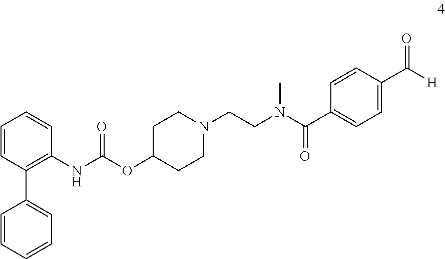 [SMILES: CN(CCN1CCC(OC(=O)Nc2ccccc2-c2ccccc2)CC1)C(=O)c1ccc(C=O)cc1]

d) contacting Compound 4 with piperidine-4-carboxamide under conditions to provide revefenacin:

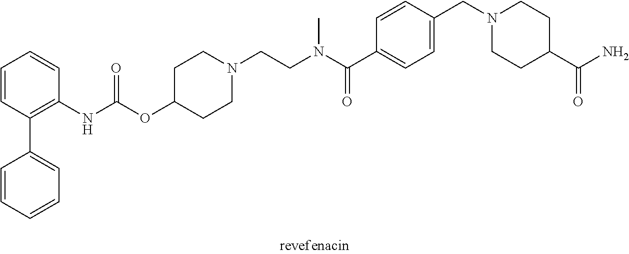 [SMILES: CN(CCN1CCC(OC(=O)Nc2ccccc2-c2ccccc2)CC1)C(=O)c1ccc(CN2CCC(C(N)=O)CC2)cc1]

and optionally:
e) crystallizing the revefenacin under conditions sufficient to provide the crystalline revefenacin Form I or crystalline revefenacin Form II.

In some embodiments, the quantity of Compound 1 added is between 67-72% by weight, between 70-72% by weight, or about 71% by weight, the quantity of Compound A.

In some embodiments, the revefenacin is crystallized. The crystalline revefenacin can be prepared as described herein or in the literature. It will be appreciated that while specific process conditions (i.e. reaction temperatures, times, mole ratios of reactants, solvents, pressures, etc.) are given, other process conditions can also be used unless otherwise stated. Generally, the reactions are conducted in a suitable inert diluent, examples of which include, but are not limited to, methanol, ethanol, isopropanol, isobutanol, ethyl acetate, acetonitrile, dichloromethane, methyl t-butyl ether, and the like, and mixtures thereof, typically containing water. Upon completion of any of the foregoing reactions, the crystalline compounds can be isolated from the reaction mixture by any conventional means such as precipitation, concentration, centrifugation and the like.

The revefenacin as disclosed herein can be a freebase or a pharmaceutically acceptable salt thereof, such as a diphosphate, monosulfate or dioxalate salt, or a solid form (e.g., crystalline form) or pharmaceutically acceptable solvate of the freebase or salt. In some embodiments, the processes disclosed herein can further comprise steps for providing such salts and solid forms. Exemplary methods for providing salts and solid forms of revefenacin can be found in WO2006099165A1, which is hereby incorporated by reference in its entirety.

### Diphosphate Salt Crystal

A diphosphate salt of revefenacin typically contains between about 1.8 and 2.2 molar equivalents of phosphate per molar equivalent of revefenacin; including between about 1.9 and 2.1 molar equivalents of phosphate per molar equivalent of revefenacin. In general, a crystalline diphosphate salt of revefenacin or a pharmaceutically acceptable solvate thereof can be prepared by contacting revefenacin with phosphoric acid. For example, the ester can be contacted with dilute aqueous phosphoric acid to form an amorphous diphosphate salt, which is then contacted with an inert diluent.

To prepare the amorphous diphosphate salt, the revefenacin is typically dissolved in aqueous phosphoric acid, diluted with water and isolated by lyophilization. Generally, this reaction is conducted at a temperature ranging from about 0 to 30° C., such as about 24° C. The ratio of milligrams of the revefenacin to microliters of 1M phosphoric acid is about 1:3 to about 1:4, or about 1:3.5. The resulting amorphous diphosphate salt is then typically contacted with about 15 mg/ml to about 25 mg/ml of inert diluent. Generally, this reaction is conducted at a temperature ranging from about 50 to 70° C., such as about 60° C. In a particular embodiment, 500 mg of the ester is taken up in 5 ml of water and 1.5 ml of 1M phosphoric acid. The pH is adjusted to approximately pH 5.3 with additional 1M phosphoric acid (equaling 2.1 molar equivalents). The clear solution is filtered, frozen and lyophilized to dryness to provide an amorphous diphosphate salt. The resulting amorphous diphosphate salt is added to an isopropanol:acetonitrile (1:1) solution, followed by the addition of water. In this reaction, the ratio of milligrams of the amorphous diphosphate salt to milliliters of isopropanol:acetonitrile is about 2:0.9 to about 2:2, or about 2:1.

Alternatively, a crystalline diphosphate salt of revefenacin can be prepared by contacting the ester with about 2.0 to about 2.1 molar equivalents of phosphoric acid. Generally, this reaction is conducted in an inert diluent at a temperature ranging from about 40 to 60° C., such as about 50° C. In a particular embodiment, the ester is added to an isopropanol:acetonitrile (1:1) solution, followed by the addition of water. After heating, phosphoric acid is added, hi this reaction, the ratio of grams of the ester to milliliters of phosphoric acid is about 5:14 to about 5:18, or about 5:16.

Both of the aforementioned processes for preparing crystalline diphosphate salts can lead to the formation of a separate, less stable, diphosphate crystal form. The more stable diphosphate crystal is the prevalent form; however, when the less stable diphosphate form is present, it can be readily converted to the more stable crystal by increasing the water content in the solvent mixture, and reheating the suspension to about 50° C. to about 70° C., typically about 60° C., for about 2 to about 6 hours, typically about 2 hours, followed by cooling to room temperature overnight with slow stirring.

### Monosulfate Salt Crystal

A monosulfate salt of revefenacin typically contains between about 0.8 and 1.2 molar equivalents of sulfate per molar equivalent of revefenacin; including between about 0.9 and 1.1 molar equivalents of sulfate per molar equivalent of revefenacin.

A crystalline monosulfate salt of revefenacin or a pharmaceutically acceptable solvate thereof can be prepared by contacting revefenacin with sulfuric acid. For example, the ester can be contacted with 1N aqueous sulfuric acid to form a monosulfate salt, which is then contacted with an inert diluent.

To prepare the monosulfate salt, revefenacin is typically dissolved in 1:1 acetonitrile:water, diluted with aqueous sulfuric acid, diluted with water and isolated by lyophilization. Generally, this reaction is conducted at a temperature ranging from about 0 to 30° C., such as about 24° C. The ratio of milligrams of the ester to milliliters of 1N sulfuric acid in water is about 325 mg/ml to about 285 mg/ml, including about 305 mg/ml. In one particular embodiment, 442 mg of the ester is taken up in 5 ml of 1:1 acetonitrile:water and 1.45 ml of 1N sulfuric acid is added slowly, while monitoring the pH. The pH is then adjusted to approximately pH 3.3. The clear solution is filtered, frozen and lyophilized to dryness to provide a monosulfate salt. The resulting monosulfate salt is then typically contacted with about 10 mg/ml to about 20 mg/ml of inert diluent. In one embodiment, this reaction is conducted at a first temperature and then at a lower second temperature, both temperatures ranging from about 50 to 80° C., such as about 60° C. to 70° C. In one embodiment, the monosulfate salt is added to an isopropanolacetonitrile (10:1) solution. In this reaction, the ratio of milligrams of the monosulfate salt to milliliters of isopropanol:acetonitrile is about 15:3 to about 15:0.8, including about 15:1. In another embodiment, this reaction is conducted at a first temperature and then at two lower temperature cycles. The first temperature ranges from about 50 to 80° C., such as about 70° C. The first lower temperature cycle varies from about 60° C. to 30° C. The second lower temperature cycle varies from about 40° C. to 30° C. In one embodiment, the monosulfate salt is added to an isopropanol:acetonitrile (10:1) solution. In this reaction, the ratio of milligrams of the monohydrate salt to milliliters of isopropanol:acetonitrile is about 161:7 to about 161:11, including about 161:9.

### Dioxalate Salt Crystal

A dioxalate salt of revefenacin typically contains between about 1.8 and 2.2 molar equivalents of oxalate per molar equivalent of revefenacin; including between about 1.9 and 2.1 molar equivalents of oxalate per molar equivalent of revefenacin.

A crystalline dioxalate salt of revefenacin or a pharmaceutically acceptable solvate thereof, can be prepared by contacting revefenacin with oxalic acid. For example, the ester can be contacted with 1M aqueous oxalic acid to form a dioxalate salt, which is then contacted with an inert diluent.

To prepare the dioxalate salt, revefenacin is typically dissolved in 1:1 acetonitrile:water, diluted with aqueous oxalic acid, diluted with water and isolated by lyophilization. Generally, this reaction is conducted at a temperature ranging from about 0 to 30° C., such as about 24° C. The ratio of milligrams of the ester to milliliters of 1M aqueous oxalic acid is about 320 mg/ml to about 280 mg/ml, including about 300 mg/ml. In one particular embodiment, 510 mg of the revefenacin is taken up in 5 ml of 1:1 acetonitrile:water and 1.7 ml of 1M aqueous oxalic acid is added slowly, while monitoring the pH. The pH is adjusted to approximately pH 3.0. The clear solution is filtered, frozen and lyophilized to dryness to provide a dioxalate salt. In one embodiment, the resulting dioxalate salt is then typically contacted with about 5 mg/ml to about 15 mg/ml of inert diluent. Generally, this reaction is conducted at a temperature ranging from about 50 to 70° C., such as about 60° C. In one embodiment, the dioxalate salt is added to an isopropanol:water (94:6) solution. In this reaction, the ratio of milligrams of the dioxalate salt to milliliters of isopropanol:water is about 10:0.8 to about 10:3, including about 10:1.

A crystalline dioxalate salt can also be prepared by forming a seed crystal of a crystalline dioxalate salt of revefenacin (synthesized as described above), forming a dioxalate salt of revefenacin by contacting revefenacin with oxalic acid and dissolving the salt in an inert diluent to form a solution, and adding the seed crystal to the solution. In one embodiment, a dioxalate salt is typically contacted with about 5 mg/ml to about 15 mg/ml of inert diluent. Generally, this reaction is conducted at a first temperature ranging from about 50 to 70° C., such as about 60^{C}C. The mixture is then cooled to a second temperature ranging from about 3 to 10° C., such as about 4° C. The seed crystal of a crystalline dioxalate salt of revefenacin is then added. In a particular embodiment, the dioxalate salt is added to an isopropanol:water (94:6) solution, in this reaction, the ratio of milligrams of the dioxalate salt to milliliters of isopropanol:water is about 150:10 to about 150:16, including about 150:13.

### Compositions Comprising Revefenacin

The processes and intermediates disclosed herein provide revefenacin, wherein the revefenacin has a unique impurity profile.

In some embodiments, the revefenacin provided by processes and intermediates (including compositions thereof) as described herein is crystalline revefenacin Form III, Form IV, or Form III and Form IV.

In some embodiments, the revefenacin provided by processes and intermediates (including compositions thereof) as described herein is crystalline revefenacin Form III. In some embodiments, crystalline revefenacin Form III (free base) can be characterized by a powder x-ray diffraction (PXRD) pattern comprising one, two, three or more diffraction peaks at °2θ values of 6.6±0.1, 13.1±0.1, 18.6±0.1, 19.7±0.1, and 20.2±0.1; and optionally further characterized by having one, two, three or more additional diffraction peaks at ° 2θ values selected from 8.8±0.1, 10.1±0.1, 11.4±0.1, 11.6±0.1, 14.8±0.1, 15.2±0.1, 16.1±0.1, 16.4±0.1, 16.9±0.1, 17.5±0.1, 18.2±0.1, 19.3±0.1, 19.9±0.1, 20.8±0.1, 21.1±0.1, 21.7±0.1, and 22.3±0.1.

Further characterizing data for crystalline revefenacin Form III can be found in WO2011008809A1, which disclosure is incorporated by reference in its entirety.

In some embodiments, the revefenacin provided by processes and intermediates (including compositions thereof) as described herein is crystalline revefenacin Form IV. In some embodiments, crystalline revefenacin Form IV (free base) can be characterized by a powder x-ray diffraction (PXRD) pattern comprising one, two, three or more diffraction peaks at °2θ values of 6.6±0.1, 13.1±0.1, 18.6±0.1, 19.7±0.1, and 20.2±0.1; and optionally further characterized by having one, two, three or more additional diffraction peaks at °2θ values selected from 10.6±0.1, 15.0±0.1, 16.0±0.1, 17.3±0.1, 17.7±0.1, 20.9±0.1, 21.4±0.1, 22.6±0.1, 24.6±0.1, and 27.8±0.1.

Further characterizing data for crystalline revefenacin Form IV can be found in WO2011008809A1, which disclosure is incorporated by reference in its entirety.

In some embodiments, provided is a pharmaceutical composition comprising crystalline revefenacin Form III and/or Form IV, wherein the pharmaceutical composition comprises not less than 90.0% revefenacin, and one or more of:
more than 0.05% of Compound 4A:

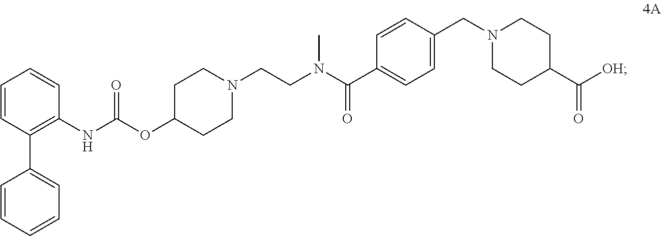 [SMILES: CN(CCN1CCC(OC(=O)Nc2ccccc2-c2ccccc2)CC1)C(=O)c1ccc(CN2CCC(C(=O)O)CC2)cc1]

more than 0.1% of Compound 4B:

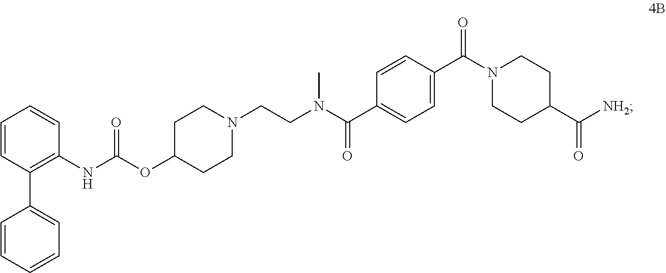 [SMILES: CN(CCN1CCC(OC(=O)Nc2ccccc2-c2ccccc2)CC1)C(=O)c1ccc(C(=O)N2CCC(C(N)=O)CC2)cc1]

more than 0.71% of Compound 4C:

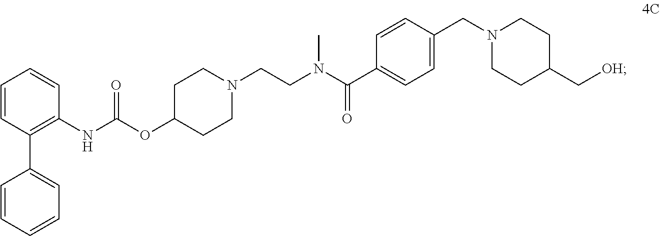 [SMILES: CN(CCN1CCC(OC(=O)Nc2ccccc2-c2ccccc2)CC1)C(=O)c1ccc(CN2CCC(CO)CC2)cc1]

more than 0.26% of Compound 4D:

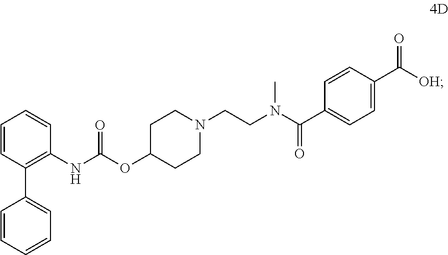 [SMILES: CN(CCN1CCC(OC(=O)Nc2ccccc2-c2ccccc2)CC1)C(=O)c1ccc(C(=O)O)cc1]

more than 0.22% of Compound 4E:

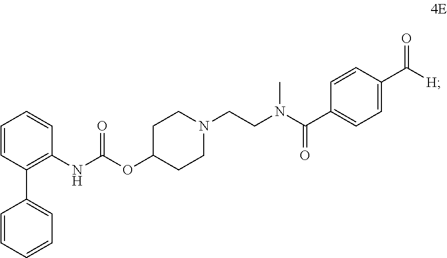

more than 0.23% of a combination of Compound 4F and Compound 4G:

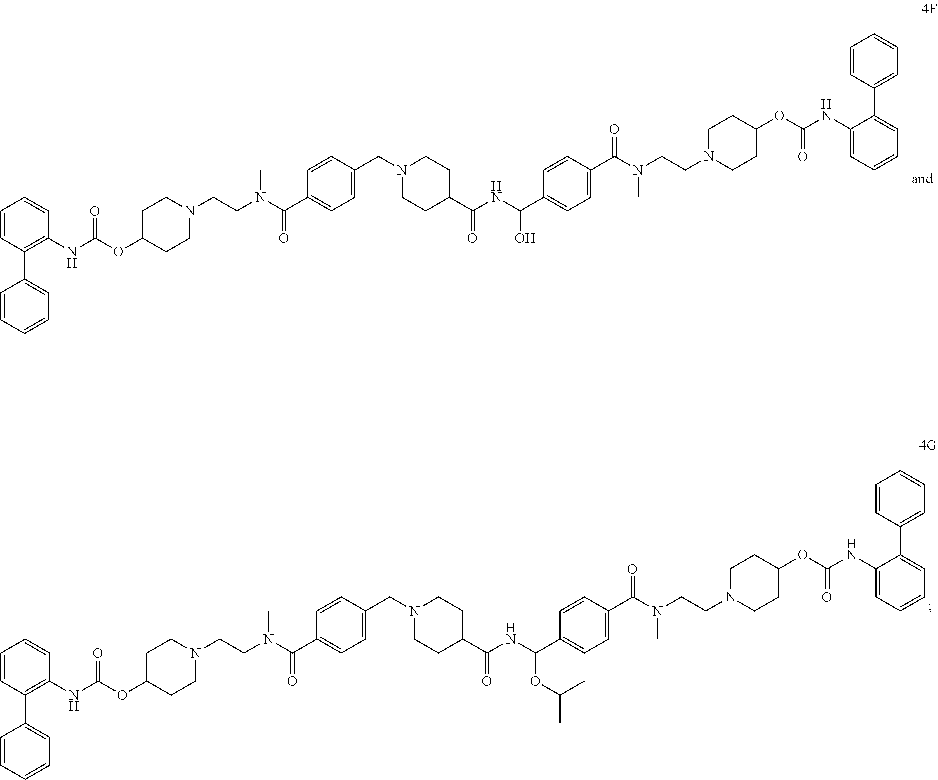 [SMILES: CN(CCN1CCC(OC(=O)Nc2ccccc2-c2ccccc2)CC1)C(=O)c1ccc(CN2CCC(C(=O)NC(O)c3ccc(C(=O)N(C)CCN4CCC(OC(=O)Nc5ccccc5-c5ccccc5)CC4)cc3)CC2)cc1 ; CC(C)OC(NC(=O)C1CCN(Cc2ccc(C(=O)N(C)CCN3CCC(OC(=O)Nc4ccccc4-c4ccccc4)CC3)cc2)CC1)c1ccc(C(=O)N(C)CCN2CCC(OC(=O)Nc3ccccc3-c3ccccc3)CC2)cc1]

more than 0.11% of Compound 4F:

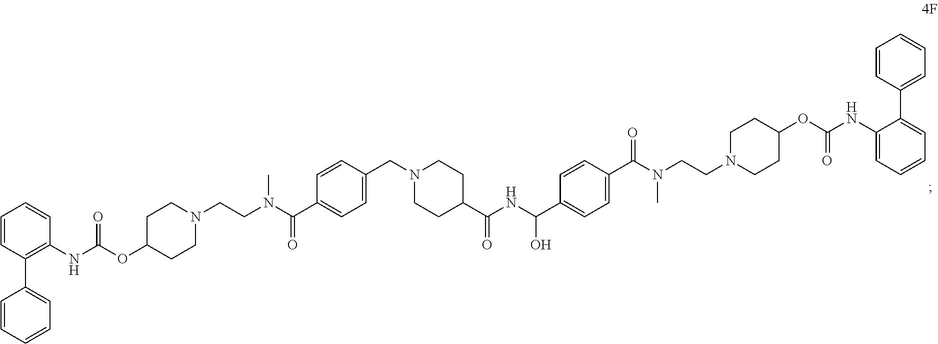 [SMILES: CN(CCN1CCC(OC(=O)Nc2ccccc2-c2ccccc2)CC1)C(=O)c1ccc(CN2CCC(C(=O)NC(O)c3ccc(C(=O)N(C)CCN4CCC(OC(=O)Nc5ccccc5-c5ccccc5)CC4)cc3)CC2)cc1]

more than 0.13% of Compound 4G:

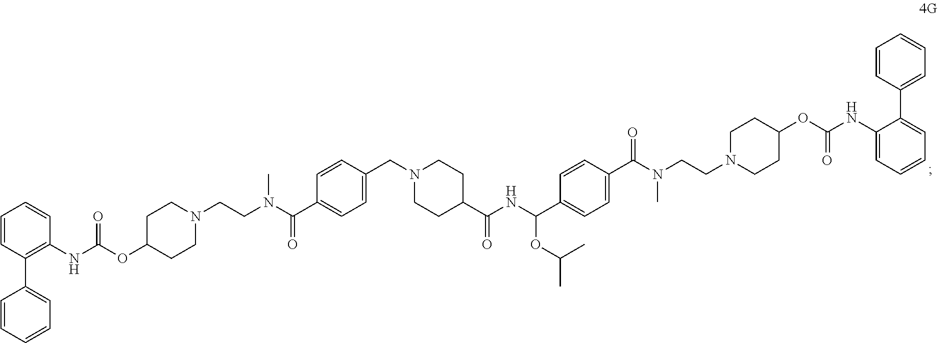 [SMILES: CC(C)OC(NC(=O)C1CCN(Cc2ccc(C(=O)N(C)CCN3CCC(OC(=O)Nc4ccccc4-c4ccccc4)CC3)cc2)CC1)c1ccc(C(=O)N(C)CCN2CCC(OC(=O)Nc3ccccc3-c3ccccc3)CC2)cc1]

or
more than 0.40% of Compound 4H:

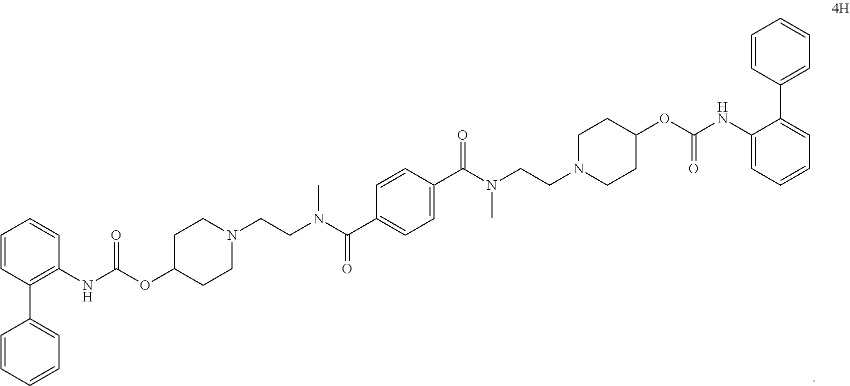 [SMILES: CN(CCN1CCC(OC(=O)Nc2ccccc2-c2ccccc2)CC1)C(=O)c1ccc(C(=O)N(C)CCN2CCC(OC(=O)Nc3ccccc3-c3ccccc3)CC2)cc1]

In some embodiments, provided is a pharmaceutical composition comprising crystalline revefenacin Form III and/or Form IV, wherein the pharmaceutical composition comprises not less than 90.0% revefenacin, and one or more of:
from 0.05% to 0.40% of Compound 4A:

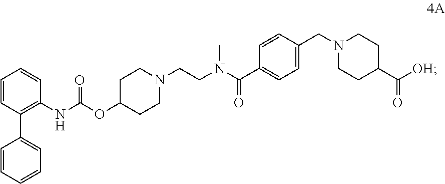 [SMILES: CN(CCN1CCC(OC(=O)Nc2ccccc2-c2ccccc2)CC1)C(=O)c1ccc(CN2CCC(C(=O)O)CC2)cc1]

from 0.1% to 0.40% of Compound 4B:

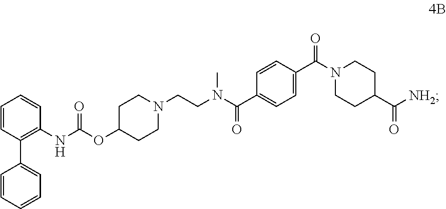 [SMILES: CN(CCN1CCC(OC(=O)Nc2ccccc2-c2ccccc2)CC1)C(=O)c1ccc(C(=O)N2CCC(C(N)=O)CC2)cc1]

from 0.71% to 1.0% of Compound 4C:

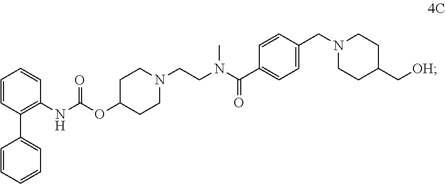 [SMILES: CN(CCN1CCC(OC(=O)Nc2ccccc2-c2ccccc2)CC1)C(=O)c1ccc(CN2CCC(CO)CC2)cc1]

from 0.26% to 0.40% of Compound 4D:

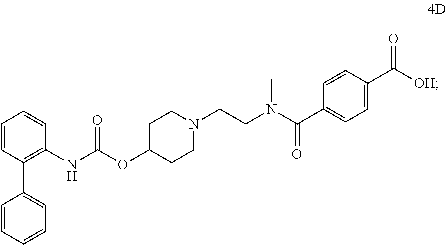 [SMILES: CN(CCN1CCC(OC(=O)Nc2ccccc2-c2ccccc2)CC1)C(=O)c1ccc(C(=O)O)cc1]

from 0.22% to 0.70% of Compound 4E:

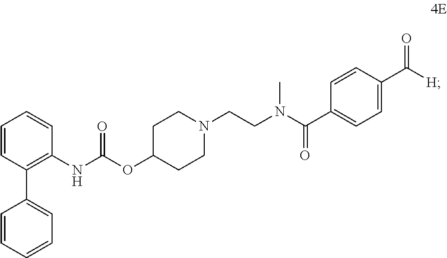

or
from 0.40% to 0.70% of Compound 4H:

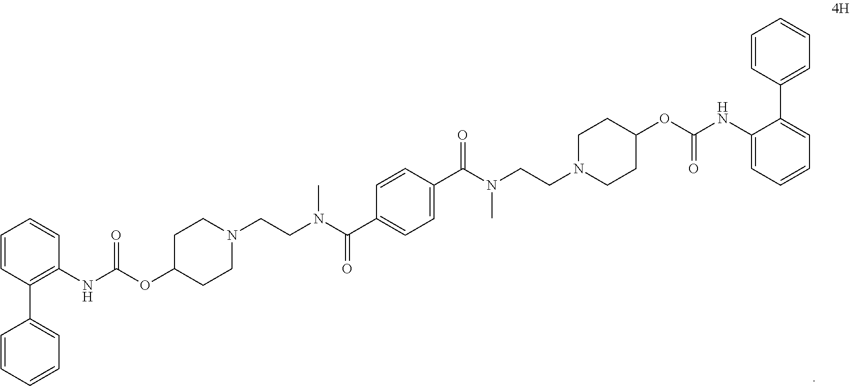 [SMILES: CN(CCN1CCC(OC(=O)Nc2ccccc2-c2ccccc2)CC1)C(=O)c1ccc(C(=O)N(C)CCN2CCC(OC(=O)Nc3ccccc3-c3ccccc3)CC2)cc1]

In some embodiments, provided is a pharmaceutical composition comprising crystalline revefenacin Form I, substantially crystalline revefenacin Form II, or substantially amorphous revefenacin. Exemplary procedures for preparing each is provided herein.

In some embodiments, provided is a pharmaceutical composition comprising revefenacin, wherein the revefenacin in the pharmaceutical composition is substantially crystalline revefenacin Form I, substantially crystalline revefenacin Form II, or substantially amorphous revefenacin, wherein the pharmaceutical composition comprises not less than 90.0% revefenacin, and not more than:
0.40% of Compound 4A:

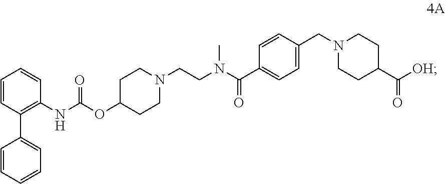 [SMILES: CN(CCN1CCC(OC(=O)Nc2ccccc2-c2ccccc2)CC1)C(=O)c1ccc(CN2CCC(C(=O)O)CC2)cc1]

0.40% of Compound 4B:

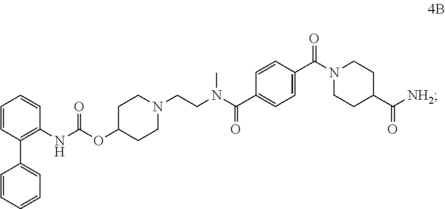 [SMILES: CN(CCN1CCC(OC(=O)Nc2ccccc2-c2ccccc2)CC1)C(=O)c1ccc(C(=O)N2CCC(C(N)=O)CC2)cc1]

1.0% of Compound 4C:

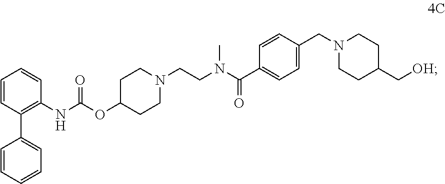 [SMILES: CN(CCN1CCC(OC(=O)Nc2ccccc2-c2ccccc2)CC1)C(=O)c1ccc(CN2CCC(CO)CC2)cc1]

0.40% of Compound 4D:

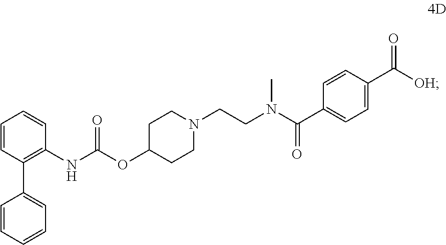 [SMILES: CN(CCN1CCC(OC(=O)Nc2ccccc2-c2ccccc2)CC1)C(=O)c1ccc(C(=O)O)cc1]

0.70% of Compound 4E:

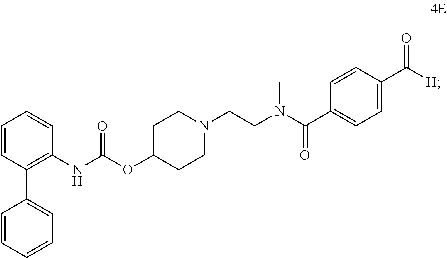

or
0.70% of Compound 4H:

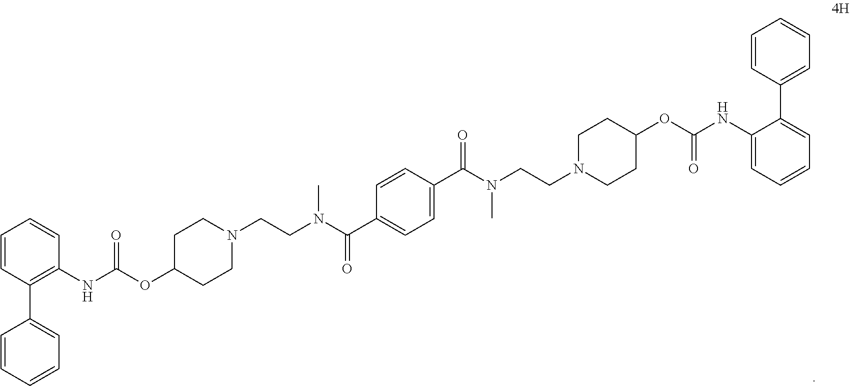 [SMILES: CN(CCN1CCC(OC(=O)Nc2ccccc2-c2ccccc2)CC1)C(=O)c1ccc(C(=O)N(C)CCN2CCC(OC(=O)Nc3ccccc3-c3ccccc3)CC2)cc1]

In the embodiments above, each “%” is “% by weight” unless otherwise indicated.

Further details regarding specific reaction conditions and other procedures are described in the Examples set forth below.

### EXAMPLES

The following Examples are provided to illustrate specific embodiments. These specific embodiments, however, are not intended to limit the scope of this disclosure in any way unless specifically indicated.

### Example 1

### Quantitative Compound 1 Assay by HPLC: Reaction of Compound 1 to 2,4-dinitrophenyihydrazone Derivative (benzyl (E)-(2-(2-(2,4-dinitrophenyl)hydrazineylidene)ethyl)(methyl)carbamate)

The amount of Compound 1 (% w/w) in the Step 2 starting material solution was determined by derivatization, followed by reversed phase HPLC analysis. As shown in the Scheme below, the derivatization consists of treatment of a solution of Compound 1 with a sufficient amount (e.g., an excess, such as a 1.5-3 fold molar excess (i.e., mol/mol)) of 2,4-dinitrophenylhydrazine to produce benzyl (E)-(2-(2-(2,4-dinitrophenyl)hydrazineylidene)ethyl)(methyl)carbamate, which is the 2,4-dinitrophenylhydrazone derivative of Compound 1.

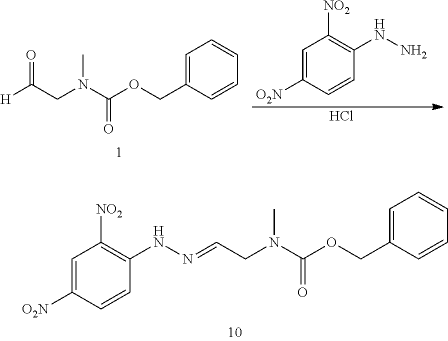

The solution of Compound 1 is sampled from the reactor after weighing the combined solution with rinse. The sample is obtained via a dip tube installed in the reactor utilizing a small amount of pressure on the headspace, vacuum in the receiving container, or a siphon/pump based sampler. Quantitation is performed against a reference standard of benzyl (E)-(2-(2-(2,4-dinitrophenyl)hydrazineylidene)ethyl)(methyl)carbamate. An exemplary procedure is provided below.

2,4-Dinitrophenylhydrazine reagent (0.55 mg/mL 2,4-dinitrophenylhydrazine in diluent) is prepared by combining 550 mg of 2,4-dinitrophenylhydrazine with 1000 mL of an acetonitrile/HCl mixture (acetonitrile:0.02N HCl, 50:50 v/v).

A reference standard is prepared by quantitatively transferring approximately 10 mg of a 2,4-dinitrophenylhydrazone derivative of Compound 1 (i.e., Compound 10) reference standard into a 100 mL volumetric flask, which is diluted to volume with acetonitrile:water, 60:40 v/v, and mixed well. The final reference standard preparation contains ˜0.1 mg/mL of Compound 10 and appears as a bright yellow solution.

The following exemplary preparation is based on a sample comprised of ˜20% w/w of Compound 1 in 2-methyltetrahydrofuran. Using a qualified positive displacement pipette or a volumetric pipette, 3.0 mL of the solution comprising Compound 1 is transferred into a tared 50 mL volumetric flask. The flask is diluted to volume with 2-methyltetrahydrofuran and mixed well to give a solution containing ˜12 mg/mL of Compound 1.

3.0 mL of the ˜12 mg/mL Compound 1 solution is transferred into a 50 mL volumetric flask, diluted to volume with 2-methyltetrahydrofuran, and mixed well to give a solution containing ˜0.7 mg/mL of Compound 1.

Into a 50 mL volumetric flask, 5.0 mL of the ˜0.7 mg/mL Compound 1 solution is transferred, and approximately 10-20 mL of 2,4-dinitrophenylhydrazine solution as prepared above (0.5 mg/mL 2,4-dinitrophenylhydrazine) is added and the solution is mixed, followed by diluting the flask to volume with derivatization reagent and mixing well. The solution is allowed to react for at least 2 hours at room temperature. The final solution preparation contains ˜0.07 mg/mL of Compound 1, which is equivalent to ˜0.13 mg/mL of Compound 10 when presuming 100% yield.

The HPLC method parameters and conditions are summarized in the Table below. The quantity of Compound 1 in 2-methyltetrahydrofuran on a % w/w basis is calculated via standard methods based on the HPLC peak area of the derivatized peak (i.e., Compound 10, r.t., =˜28 mins).

| Parameter | Condition | | |
|---|---|---|---|
| Column | Advanced Materials Halo RP-Amide | | |
| | (150 mm × 4.6 mm, 2.7 μm) | | |
| | or equivalent | | |
| Flow Rate | 1.2 mL/min | | |
| Injection Volume | 18 μL | | |
| Column | 30° C. | | |
| Temperature | | | |
| Autosampler | Ambient | | |
| Temperature | | | |
| Detection | UV, 362 nm | | |
| Run Time | 58 min | | |
| Mobile Phase A | acetonitrile:water:trifluoroacetic | | |
| | acid (1:99:0.05 v/v/v) | | |
| Mobile Phase B | acetonitrile:water:trifluoroacetic | | |
| | acid (90:10:0.05 v/v/v) | | |
| | Time | % Mobile | % Mobile |
| | (minutes) | Phase A | Phase B |
| Gradient | 0.0 | 80 | 20 |
| | 48.0 | 44 | 56 |
| | 48.1 | 0 | 100 |
| | 53.0 | 0 | 100 |
| | 53.1 | 80 | 20 |
| | 58.0 | 80 | 20 |

### Example 2—Exemplary Procedure for Compound 2

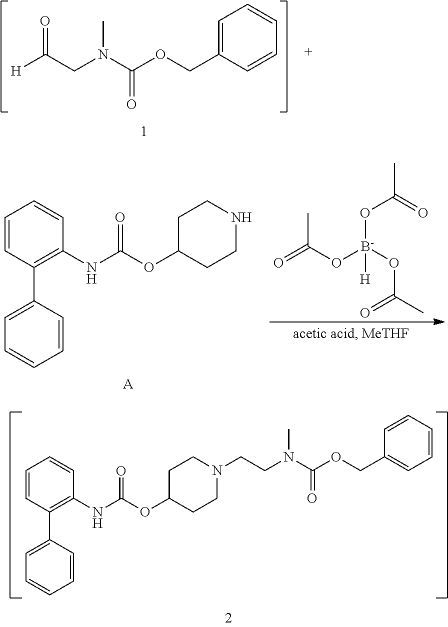

The notation ‘X’ refers to a multiplier representing the weight of Compound A. Biphenyl-2-yl-carbamic acid piperidin-4-yl ester (Compound A) (1.0×), sodium triacetoxyborohydride (1.65×), and MeTHF (3.2×) was added to a reactor. The mixture was stirred while cooling to 8° C. Glacial acetic acid (0.2×) was added while maintaining the temperature below 15° C., then the solution was cooled to about 0° C. Compound 1 (0.70×) in MeTHF was added slowly over 0.5-1.5 hours while maintaining the temperature below 10° C. The solution was further stirred for 2 hours at 8° C., until reaction was confirmed complete by HPLC. Then 7% wt/wt sodium bicarbonate aqueous solution (8.71×) was added, while maintaining the temperature below 30° C. The mixture was stirred for 15 min and the layers were allowed to separate for 30 min. The aqueous layer was discarded and to the organic layer was added 7% wt/wt sodium bicarbonate aqueous solution (8.71×) while maintaining the temperature below 30° C. The mixture was stirred for 15 min and the layers were allowed to separate for 30 min. The layers were separated and the aqueous layer was discarded, resulting in isolation of Compound 2 in the organic layer.

### Example 3

### Procedure A: (2,2-Dimethoxyethyl)methylcarbamic Acid Benzyl Ester

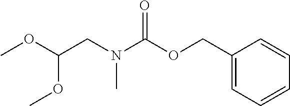 [SMILES: COC(CN(C)C(=O)OCc1ccccc1)OC]

K₂CO₃ (8.4 kg, 60 mol, 1.8 eq.) and H₂O (49.3 kg, 2.6 volumes) were placed in the reaction vessel and stirred. N-methylaminoacetaldehyde dimethylacetal (6.5 kg, 54 mol, 1.6 eq) and MeTHF (20.2 kg, 2.9 volumes) were added. The resulting mixture was cooled to 5° C. Benzyl chloroformate (6.8 kg, 37.6 mol, 1.1 eq.) was added over a period of about 30 minutes, while maintaining the temperature below 10° C. The feed line was rinsed with MeTHF (4.3 kg). The mixture was then maintained at 5° C. and stirred for 1 hour. The layers were separated and the organic layer was washed with 1N HCl (14.3 kg, 11.7 mol, 1.4 volumes) and used directly in the next step.

### Procedure B: Methyl-(2-oxoethyl)carbamic Acid Benzyl Ester (Compound 1)

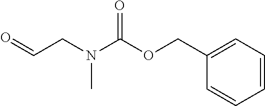

The mixture from the previous step was combined with water (23.4 kg, 2.9 volumes) and 30% hydrochloric acid (13.1 kg, 107.7 mol, 1.1 volumes). Water (5.1 kg) was used to rinse the feed line. The temperature was adjusted to 25-30° C., and the reaction was run for 16-24 hours. A 25% NaOH solution (11.8 kg, 71.1 mol, 2.2 eq.) was added to the solution to adjust the pH and obtain phase separation.

The layers were separated and the aqueous layer was back-extracted with MeTHF (10.0 kg, 1.1 volumes). The aqueous layer was discarded and the organic layers were combined. MeTHF (4.4 kg) was used to rinse the feed line. The organics were washed with a saturated NaHCO₃ solution (14.6 kg, 15.6 mol, 1.1 volumes). The layers were separated and the organic layer was dried over Na₂SO₄ (2.5 kg, 17.6 mol) for 60-90 minutes. The drying agent was filtered off and the remaining solids were washed with MeTHF (8.8 kg, 1 volume). The reaction vessel was washed with water and MeOH before continuing with the next step.

### Procedure C: Compound 2 is Used in the Following Procedure as Prepared in Example 2

### Procedure D: Biphenyl-2-yl-carbamic acid 1-(2-methylaminoethyl)piperidin-4-yl Ester (Compound 3)

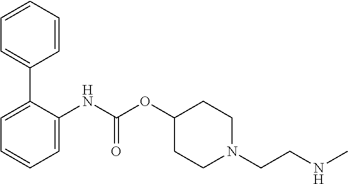 [SMILES: CNCCN1CCC(OC(=O)Nc2ccccc2-c2ccccc2)CC1]

10% palladium on carbon (0.4 kg, 0.03 wt %, Degussa type 101 NE/W) was added to the reaction mixture. A hydrogenation reaction was performed to remove the benzyloxycarbonyl protective group of Compound 2 with reaction conditions at 30±5° C. and 4 bar pressure. The reaction was run until completion. The mixture was then filtered and the filter cake was washed with MeOH (8.0 kg, 1.0 volume). The reaction was continued in a clean vessel, which was charged with the product solution (in MeTHF/MeOH) from the hydrogenation reaction. 3-Mercaptopropyl silica (0.6 kg, 0.07 wt %, Silicycle) was added. MeOH (4.8 kg) was used to rinse the feed line. The reaction mixture was stirred for 14-72 hours at 25±5° C. Activated carbon (0.7 kg, 0.07 wt %) was added and the mixture stirred for 30 minutes. The mixture was filtered and the filter cake was washed with MeOH (1.0 volume). The reaction was continued in a clean vessel, which was charged with the product solution (in MeTHF/MeOH), and MeOH (4.2 kg) was used to rinse the feed line. The mixture was heated to 40-45° C. and a vacuum distillation was performed to bring the final volume to 5.6 volumes (removal of methanol).

2-propanol (40.2 kg, 5.0 volumes) was added and distillation continued until the volume was reduced to 2.5 volumes. The solids were then isolated by filtration and washed with MTBE (1.5 volumes) to yield the product as a wet cake (8.6 kg, 96.8% purity). The cake was charged to the reaction vessel and additional 2-propanol (1.9 volumes) was added. The mixture was warmed to 40±5° C., and maintained at that temperature for 2 hours. The mixture was then slowly cooled over a minimum of 4 hours to 20° C., then actively cooled to 5-10° C., followed by stirring for 2 hours. The product was filtered and the resulting cake washed with MTBE (1.0 volume). The solids were then dried under atmospheric conditions to yield the title compound (6.6 kg, 98.5% purity).

### Procedure E: Biphenyl-2-yl-carbamic acid 1-{2-[(4-formylbenzoyl) methylamino]ethyl}piperidin-4-yl Ester (Compound 4)

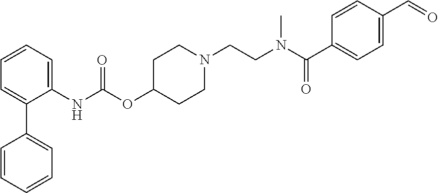 [SMILES: CN(CCN1CCC(OC(=O)Nc2ccccc2-c2ccccc2)CC1)C(=O)c1ccc(C=O)cc1]

4-Carboxybenzaldehyde (9 g, 60 mmol, 1.0 eq.) and biphenyl-2-yl-carbamic acid 1-(2-methylaminoethyl)piperidin-4-yl ester (Compound 3) (21.2 g, 60 mmol, 1.0 eq.) were combined in MeTHF (115 mL). The mixture was stirred for 0.5 hours, forming a thick slurry. Additional MeTHF (50 mL) was added to form a free-flowing slurry. 4-(4,6-dimethoxy-1,3,5-triazin-2-yl)-4-methylmorpholinium chloride (18 g, 63 mmol, 1.1 eq., 97% pure) was added in two portions and the funnel rinsed with additional MeTHF (50 mL). The mixture was stirred at room temperature overnight. MeCN (50 mL) was added and the mixture was filtered. The solids were washed with MeTHF (30 mL). The filtrate and washes were combined and a saturated NaHCO₃ solution (100 mL) was added and stirred for 10 minutes. The layers were separated and a saturated NaCl solution (100 mL) was added and stirred for 10 minutes. The layers were separated and the aqueous layer discarded. The resulting solution was concentrated under reduced pressure and held at room temperature for three days, then used directly in the next step.

### Procedure F: Revefenacin (Non-Isolated Form)

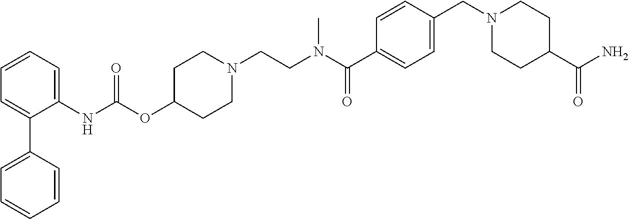 [SMILES: CN(CCN1CCC(OC(=O)Nc2ccccc2-c2ccccc2)CC1)C(=O)c1ccc(CN2CCC(C(N)=O)CC2)cc1]

Isonipecotamide (15.4, 120 mmol, 2.0 eq.) and IPA (200 mL) were added to the solution of biphenyl-2-yl-carbamic acid 1-{2-[(4-formylbenzoyl)methylamino]ethyl}piperidin-4-yl ester from the previous step. Liquid (200 mL) was distilled off and additional IPA (400 mL) was added under reduced pressure at 60° C. Liquid (400 mL) was distilled off over a period of 1.5 hours and additional IPA (600 mL) was added. Liquid (100 mL) was distilled off and the remaining solution was cooled to 30° C. to yield a hazy white mixture, which was then added to Na₂SO₄ (18 g). The flask was rinsed with IPA (100 mL) and added to the solution. The resulting mixture was cooled to room temperature and AcOH (20 mL, 360 mmol, 6.0 eq.) was added. The mixture was cooled to 18° C. with an ice bath and NaHB(OAc)₃ (38.2 g, 180 mmol, 3.0 eq.) was added over 5 minutes. The mixture was allowed to warm up to 25° C. and was maintained at that temperature for 2 hours. Solvent was removed under reduced pressure, and the remaining material was used directly in the next step.

### Procedure G: Biphenyl-2-yl-carbamic acid 1-{2-[(4-carbamoylbenzoyl) methylamino]ethyl}piperidin-4-yl ester (Revefenacin Isolated Solid)

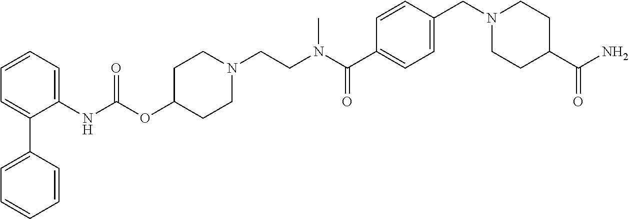 [SMILES: CN(CCN1CCC(OC(=O)Nc2ccccc2-c2ccccc2)CC1)C(=O)c1ccc(CN2CCC(C(N)=O)CC2)cc1]

iPrOAc (300 mL) was added to the material, followed by the addition of water (200 mL). The pH of the solution was adjusted to pH 1 with 3N HCl (˜150 mL). The layers were separated and the organic layer was discarded. The aqueous layer was collected, and iPrOAc (300 mL) was added. The pH of the solution was adjusted to basic pH with 50 wt % NaOH (˜100 mL). The resulting mixture was stirred for 15 minutes and the layers were separated. The organic layer was filtered and seeded with micronized crystalline freebase of biphenyl-2-yl-carbamic acid 1-{2-[(4-carbamoylbenzoyl) methylamino]ethyl}piperidin-4-yl ester (Form III; prepared as described in U.S. Patent Application Publication No. 2011/0015163 to Woollam) and stirred overnight at room temperature to yield a white slurry. Stirring was continued for 8 hours at room temperature and for 16 hours at 5° C. (cold room). The mixture was slowly filtered under pressure. The cake was washed with cold iPrOAc (2×20 mL) and dried under nitrogen to yield a white solid (27.5 g). The material was further dried in a vacuum oven at 30° C. for 24 hours to yield 25.9 g.

### Procedure H: Crystalline Freebase of Biphenyl-2-yl-carbamic Acid 1-{2-[(4-carbamoylbenzoyl)methylamino]ethyl}piperidin-4-yl Ester (Revefenacin Form III)

The white solid (5 g, 60 mmol, 1.0 eq.) was dissolved in toluene (75 mL) and the resulting mixture was heated to 82° C. to yield a clear solution. The solution was filtered. The solids were washed with toluene (2×5 mL), and the filtrate and washes were combined. The mixture was cooled to 60° C. and seeded with micronized crystalline freebase of biphenyl-2-yl-carbamic acid 1-{2-[(4-carbamoylbenzoyl)methylamino]ethyl}piperidin-4-yl ester (Form III; prepared as described in Example 3 in U.S. Patent Application Publication No. 2011/0015163 to Woollam). The mixture was maintained at 55° C. for 2 hours, then cooled to room temperature on an oil bath overnight (˜16 hours). The resulting slurry was then filtered and the cake was dried for 3 hours to yield a solid white material (4.6 g). The material was further dried in a vacuum oven at 30° C. for 24 hours (exhibited no further weight loss) to yield the title compound (4.6 g).

The product was analyzed by powder x-ray diffraction, differential scanning calorimetry and thermal gravimetric analysis, and was determined to be the crystalline freebase (Form III) of biphenyl-2-ylcarbamic acid 1-(2-{[4-(4-carbamoylpiperidin-1-ylmethyl)benzoyl]methylamino}ethyl)piperidin-4-yl ester described in U.S. Patent Application Publication No. 2011/0015163 to Woollam.

### Procedure I: Crystalline Freebase of Biphenyl-2-yl-carbamic Acid 1-{2-[(4-carbamoylbenzoyl)methylamino]ethyl}piperidin-4-yl Ester (Revefenacin Form IV)

Revefenacin Form IV can be prepared according to Example 4 of U.S. Patent Application Publication No. 2011/0015163 to Woollam. Briefly, biphenyl-2-ylcarbamic acid 1-(2-{[4-(4-carbamoylpiperidin-1-ylmethyl)benzoyl]methylamino}ethyl)piperidin-4-yl ester was dissolved in MeCN to yield a viscous oily pale yellow material. Additional MeCN (5 mL) was added to dilute the material. The solution was seeded with Revefenacin Form III and stirred at room temperature for 90 minutes. A large amount of white precipitate (small crystals) was observed. The slurry was analyzed under a polarized light microscope and found to be birefringent.

Additional MeCN (3 mL) was added and the slurry was placed in a Metz Syn10 block to thermocycle (0-40° C. in one hour blocks) at 800 rpm overnight. The Metz Syn10 is a 10 position parallel reaction station that is static. Agitation of the solution/slurry was by a cross magnetic stirrer bar. The shaker block was a separate piece of equipment that was heated and cooled by an external Julabo bath. The material was removed at 0° C. It was observed that the slurry had settled out, leaving a pale yellow solution above the white precipitate. The slurry was stirred and placed back in the shaker block to thermocycle. The material was removed at 40° C., and stirred at a high agitation rate at room temperature for 80 minutes. The slurry was again analyzed and found to be birefringent. The filter cake was isolated by vacuum filtration using a sinter funnel. MeCN (3 mL) was used to wet the filter paper and the filter cake was washed with MeCN prior to filtration. The cake was deliquored under vacuum for 40 minutes to yield a flowing white powder. The material was placed in a piston dryer at 40° C. for 65 hours, to yield the title crystalline compound as a white powder (99.6% purity).

### Example 4

The following Example provides exemplary procedures for providing crystalline forms of revefenacin.

### Crystalline Freebase (Form I)

50.4 mg of revefenacin was dissolved in 0.144 ml of H₂O:ACN (1:1). The suspension was left in a vial (cap loosely placed on top) to allow for a slower evaporation time. The vial was refrigerated at 4° C. for 6 days. A precipitate was visible after 2 days. The solid was filtered and placed on a high vacuum line to remove all solvent and give the title compound as a white solid (27.8 mg, 55.2% yield).

### Crystalline Freebase (Form I)

230 mg of revefenacin was dissolved in 0.2 ml of H₂O:ACN (1:1), using slight heat. The mixture was then heated in a 70° C. water bath for 2 hours. The heat was turned off and the mixture was allowed to cool to room temperature, then refrigerated at 4° C. for 1 hour. 50 μL of water was then added (oiled out), followed by the addition of 40 μL of ACN to get the sample back into solution. Seeds (crystalline material from Example 8) were added under slow stirring at room temperature. Crystals started to form, and the mixture was allowed to sit overnight, with slow stirring. The next day, a heat cool cycle was applied (30° C. for 10 minutes, 40° C. for 10 minutes, then 50° C. for 20 minutes). The heat was turned off and the mixture allowed to cool overnight, with slow stirring. The next day, a second heat/cool cycle was applied (60° C. for 1 hour, with dissolving observed at 70° C.). The heat was turned off and the mixture allowed to cool overnight, with slow stirring. The next day, crystals were present and a third heat cool cycle was applied (60° C. for 3 hours). The heat was turned off and the mixture allowed to cool overnight, with slow stirring. The next day, a heat cool cycle was applied (60° C. for 3 hours, slow cool, then 60° C. for 3 hours). The heat was turned off and the mixture allowed to cool overnight, with slow stirring. After 3 days, the solid was filtered and placed on a high vacuum line to remove all solvent and give the title compound.

### Crystalline Freebase (Form II)

70 mg of revefenacin was dissolved in 0.1 mL ACN. After addition of 0.3 ml MTBE, the solution appeared cloudy. An additional 50 μL of ACN was added to clarify the solution (155 mg/mL ACN:MTBE=1:2). The mixture was left in the vial and capped. Crystals appeared by the next day. The solid was then filtered and placed on a high vacuum line to remove all solvent and give the title compound.

While the present disclosure has been described with reference to specific aspects or embodiments thereof, it will be understood by those of ordinary skilled in the art that various changes can be made or equivalents can be substituted without departing from the true spirit and scope of the disclosure.

Additionally, to the extent permitted by applicable patent statues and regulations, all publications, patents and patent applications cited herein are hereby incorporated by reference in their entirety to the same extent as if each document had been individually incorporated by reference herein.

1. A composition comprising Compound 3: 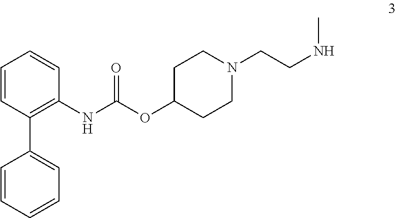 [SMILES: CNCCN1CCC(OC(=O)Nc2ccccc2-c2ccccc2)CC1]
wherein the composition comprises less than 0.3% Compound 1A: 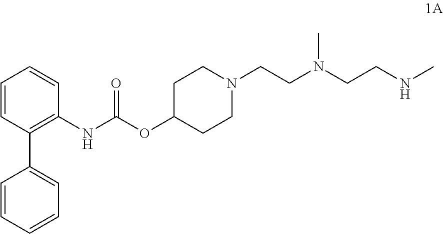 [SMILES: CNCCN(C)CCN1CCC(OC(=O)Nc2ccccc2-c2ccccc2)CC1]
or less than 0.55% Compound 1B:

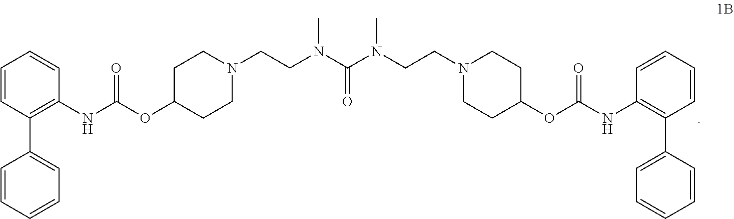 [SMILES: CN(CCN1CCC(OC(=O)Nc2ccccc2-c2ccccc2)CC1)C(=O)N(C)CCN1CCC(OC(=O)Nc2ccccc2-c2ccccc2)CC1]

2. The composition of claim 1, wherein the composition comprises less than 0.67% Compound A:

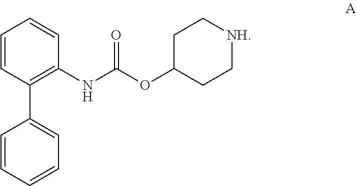 [SMILES: O=C(Nc1ccccc1-c1ccccc1)OC1CCNCC1]

3. A process for preparing the composition of claim 1, comprising:
a) adding a composition comprising a quantity of Compound 1 to a composition comprising Compound A, such that the quantity of Compound 1 added is less than 73% by weight the quantity of Compound A: 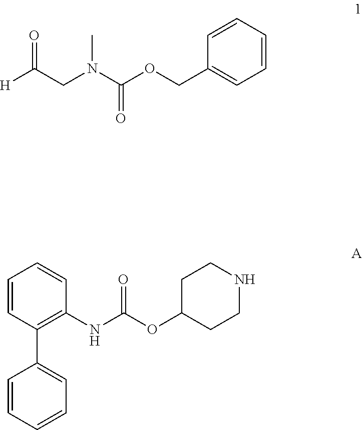 [SMILES: CN(CC=O)C(=O)OCc1ccccc1 ; O=C(Nc1ccccc1-c1ccccc1)OC1CCNCC1]
under conditions sufficient to provide a composition comprising Compound 2: 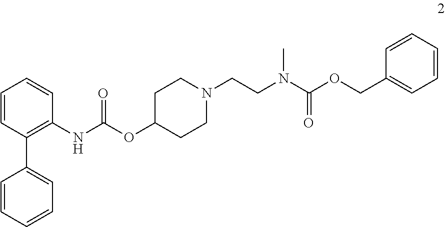 [SMILES: CN(CCN1CCC(OC(=O)Nc2ccccc2-c2ccccc2)CC1)C(=O)OCc1ccccc1]
and
b) hydrogenating the composition comprising Compound 2 under conditions sufficient to provide the composition comprising Compound 3.

4. The process of claim 3, wherein the quantity of Compound 1 added is between 67-72% by weight the quantity of Compound A.

5. The process of claim 3 wherein prior to step a) the process comprises determining the concentration of Compound 1 in the composition comprising Compound 1.

6. The process of claim 3, wherein the determining comprises reacting an aliquot of the composition comprising Compound 1 with 2,4-dinitrophenylhydrazine to provide benzyl (E)-(2-(2-(2,4-dinitrophenyl)hydrazineylidene)ethyl)(methyl)carbamate (Compound 10): 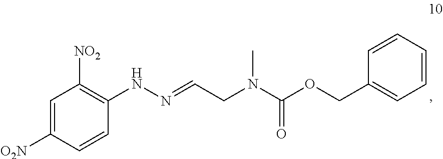 [SMILES: CN(C/C=N/Nc1ccc([N+](=O)[O-])cc1[N+](=O)[O-])C(=O)OCc1ccccc1]
and determining the quantity of benzyl (E)-(2-(2-(2,4-dinitrophenyl)hydrazineylidene)ethyl)(methyl)carbamate by HPLC.

7. A composition comprising Compound 4: 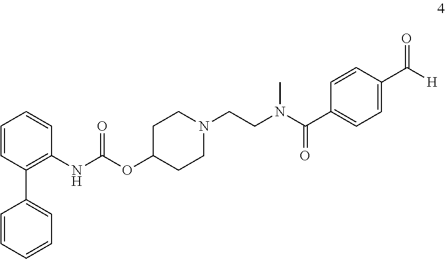 [SMILES: CN(CCN1CCC(OC(=O)Nc2ccccc2-c2ccccc2)CC1)C(=O)c1ccc(C=O)cc1]
wherein the composition comprises less than 0.55% Compound 3A: 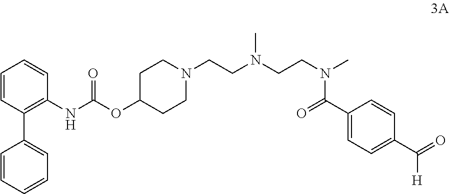 [SMILES: CN(CCN1CCC(OC(=O)Nc2ccccc2-c2ccccc2)CC1)CCN(C)C(=O)c1ccc(C=O)cc1]
or less than 0.55% Compound 1B:

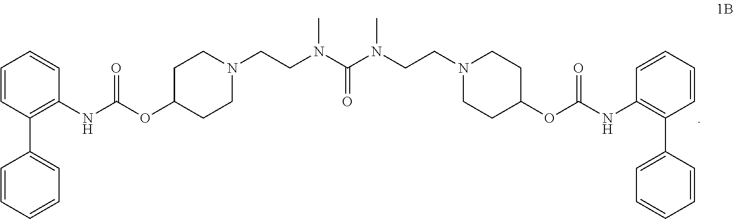 [SMILES: CN(CCN1CCC(OC(=O)Nc2ccccc2-c2ccccc2)CC1)C(=O)N(C)CCN1CCC(OC(=O)Nc2ccccc2-c2ccccc2)CC1]

8. A process for preparing a composition comprising Compound 4: 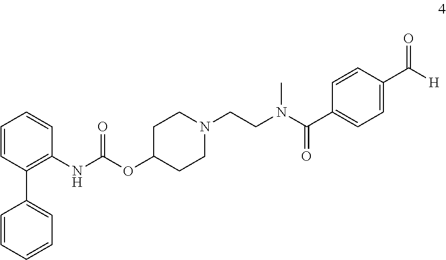 [SMILES: CN(CCN1CCC(OC(=O)Nc2ccccc2-c2ccccc2)CC1)C(=O)c1ccc(C=O)cc1]
wherein the composition comprises less than 0.55% Compound 3A: 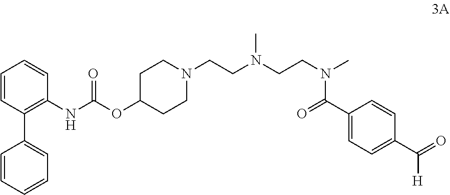 [SMILES: CN(CCN1CCC(OC(=O)Nc2ccccc2-c2ccccc2)CC1)CCN(C)C(=O)c1ccc(C=O)cc1]
or less than 0.55% Compound 1B: 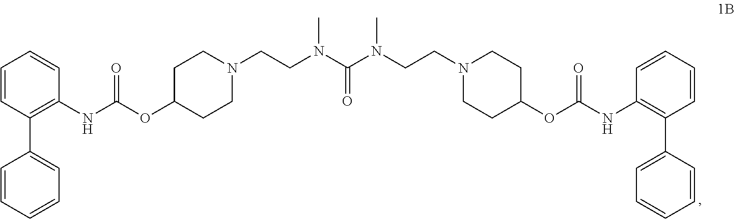 [SMILES: CN(CCN1CCC(OC(=O)Nc2ccccc2-c2ccccc2)CC1)C(=O)N(C)CCN1CCC(OC(=O)Nc2ccccc2-c2ccccc2)CC1]
wherein the process comprises contacting the composition comprising Compound 3 according to claim 1, with 4-formylbenzoic acid under conditions sufficient to provide the composition comprising Compound 4.

9. A process for preparing crystalline revefenacin Form I, crystalline revefenacin Form II, or amorphous revefenacin, said process comprising:
i) contacting the composition comprising Compound 3 according to claim 1, with 4-formylbenzoic acid under conditions sufficient to provide Compound 4: 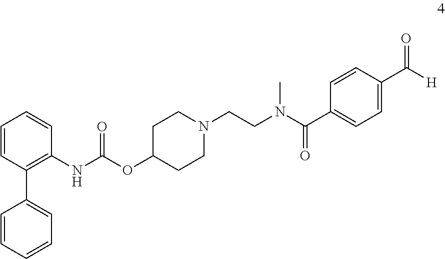 [SMILES: CN(CCN1CCC(OC(=O)Nc2ccccc2-c2ccccc2)CC1)C(=O)c1ccc(C=O)cc1]
ii) contacting Compound 4 with piperidine-4-carboxamide under conditions sufficient to provide revefenacin: 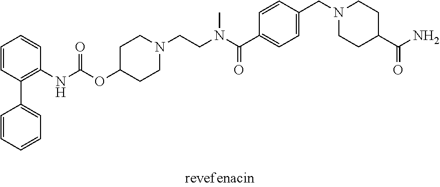 [SMILES: CN(CCN1CCC(OC(=O)Nc2ccccc2-c2ccccc2)CC1)C(=O)c1ccc(CN2CCC(C(N)=O)CC2)cc1]
and optionally:
iii) crystallizing the revefenacin under conditions sufficient to provide the crystalline revefenacin Form I or crystalline revefenacin Form II.

10. A process for preparing crystalline revefenacin Form I, crystalline revefenacin Form II, or amorphous revefenacin, said process comprising:
i) contacting the composition of claim 7 with piperidine-4-carboxamide under conditions sufficient to provide revefenacin: 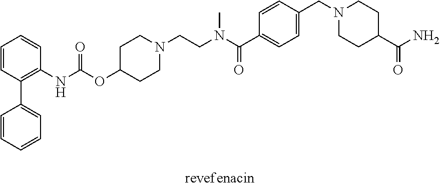 [SMILES: CN(CCN1CCC(OC(=O)Nc2ccccc2-c2ccccc2)CC1)C(=O)c1ccc(CN2CCC(C(N)=O)CC2)cc1]
and optionally:
ii) crystallizing the revefenacin under conditions sufficient to provide the crystalline revefenacin Form I or crystalline revefenacin Form II.

11. A process for preparing crystalline revefenacin Form I, crystalline revefenacin Form II, or amorphous revefenacin, said process comprising:
a) adding a composition comprising Compound 1 to a composition comprising Compound A: 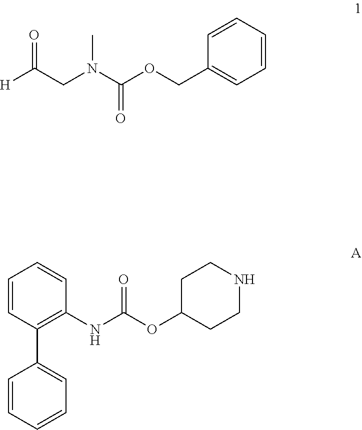 [SMILES: CN(CC=O)C(=O)OCc1ccccc1 ; O=C(Nc1ccccc1-c1ccccc1)OC1CCNCC1]
under conditions sufficient to provide a composition comprising Compound 2: 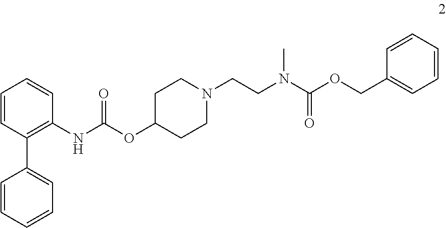 [SMILES: CN(CCN1CCC(OC(=O)Nc2ccccc2-c2ccccc2)CC1)C(=O)OCc1ccccc1]
wherein the quantity of Compound 1 added is determined by reacting an aliquot of the composition comprising Compound 1 with 2,4-dinitrophenylhydrazine under conditions sufficient to provide benzyl (E)-(2-(2-(2,4-dinitrophenyl)hydrazineylidene)ethyl)(methyl)carbamate (Compound 10): 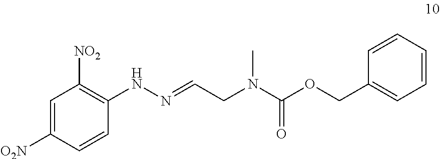 [SMILES: CN(C/C=N/Nc1ccc([N+](=O)[O-])cc1[N+](=O)[O-])C(=O)OCc1ccccc1]
and determining the quantity of benzyl (E)-(2-(2-(2,4-dinitrophenyl)hydrazineylidene)ethyl)(methyl)carbamate by HPLC;
b) hydrogenating the composition comprising Compound 2 under conditions sufficient to provide Compound 3: 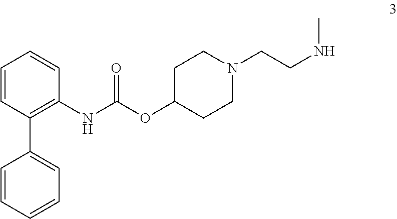 [SMILES: CNCCN1CCC(OC(=O)Nc2ccccc2-c2ccccc2)CC1]
c) contacting Compound 3 with 4-formylbenzoic acid under conditions sufficient to provide Compound 4: 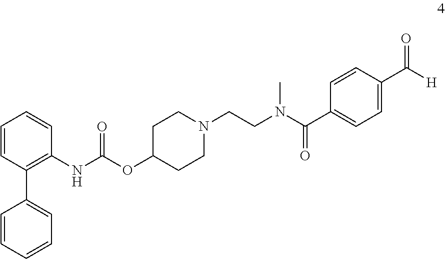 [SMILES: CN(CCN1CCC(OC(=O)Nc2ccccc2-c2ccccc2)CC1)C(=O)c1ccc(C=O)cc1]
d) contacting Compound 4 with piperidine-4-carboxamide under conditions sufficient to provide revefenacin: 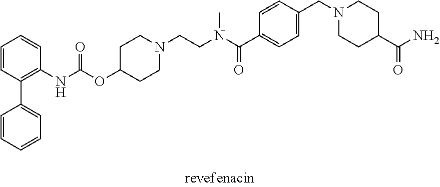 [SMILES: CN(CCN1CCC(OC(=O)Nc2ccccc2-c2ccccc2)CC1)C(=O)c1ccc(CN2CCC(C(N)=O)CC2)cc1]
and optionally:
e) crystallizing the revefenacin under conditions sufficient to provide the crystalline revefenacin Form I or crystalline revefenacin Form II.

12. A process for preparing crystalline revefenacin Form I, crystalline revefenacin Form II, or amorphous revefenacin, said process comprising:
a) contacting a composition comprising Compound 3: 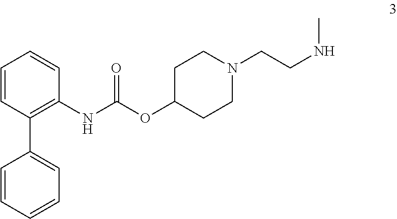 [SMILES: CNCCN1CCC(OC(=O)Nc2ccccc2-c2ccccc2)CC1]
wherein the composition comprises less than 0.3% Compound 1A: 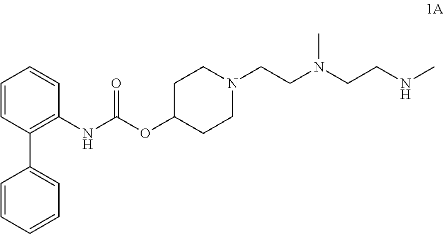 [SMILES: CNCCN(C)CCN1CCC(OC(=O)Nc2ccccc2-c2ccccc2)CC1]
or less than 0.55% Compound 1B: 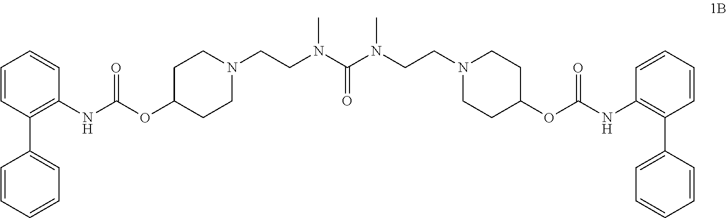 [SMILES: CN(CCN1CCC(OC(=O)Nc2ccccc2-c2ccccc2)CC1)C(=O)N(C)CCN1CCC(OC(=O)Nc2ccccc2-c2ccccc2)CC1]
or less than 0.67% Compound A: 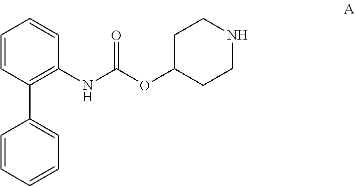 [SMILES: O=C(Nc1ccccc1-c1ccccc1)OC1CCNCC1]
with 4-formylbenzoic acid to provide Compound 4: 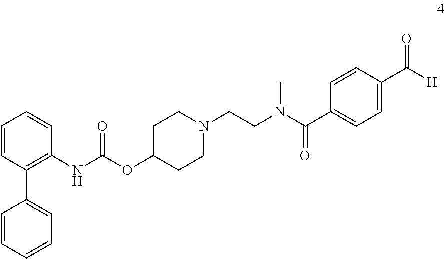 [SMILES: CN(CCN1CCC(OC(=O)Nc2ccccc2-c2ccccc2)CC1)C(=O)c1ccc(C=O)cc1]
b) contacting Compound 4 with piperidine-4-carboxamide under conditions sufficient to provide revefenacin: 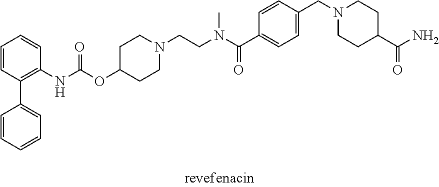 [SMILES: CN(CCN1CCC(OC(=O)Nc2ccccc2-c2ccccc2)CC1)C(=O)c1ccc(CN2CCC(C(N)=O)CC2)cc1]
and optionally:
c) crystallizing the revefenacin under conditions sufficient to provide the crystalline revefenacin Form I or crystalline revefenacin Form II.

13. A process for preparing crystalline revefenacin Form I, crystalline revefenacin Form II, or amorphous revefenacin, said process comprising:
a) adding a composition comprising Compound 1 to a composition comprising Compound A, such that the quantity of Compound 1 added is less than 73% by weight the quantity of Compound A: 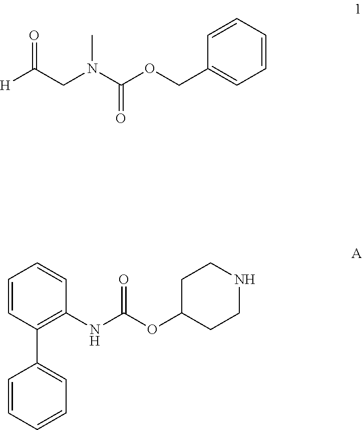 [SMILES: CN(CC=O)C(=O)OCc1ccccc1 ; O=C(Nc1ccccc1-c1ccccc1)OC1CCNCC1]
under conditions sufficient to provide Compound 2: 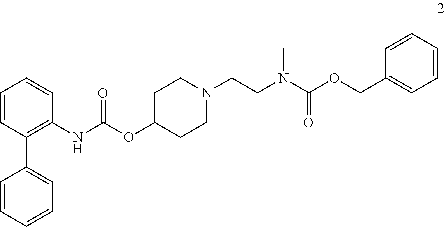 [SMILES: CN(CCN1CCC(OC(=O)Nc2ccccc2-c2ccccc2)CC1)C(=O)OCc1ccccc1]
b) hydrogenating the composition comprising Compound 2 under conditions to provide Compound 3: 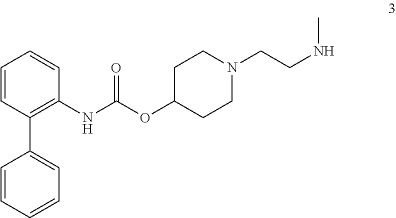 [SMILES: CNCCN1CCC(OC(=O)Nc2ccccc2-c2ccccc2)CC1]
c) contacting Compound 3 with 4-formylbenzoic acid, to provide Compound 4: 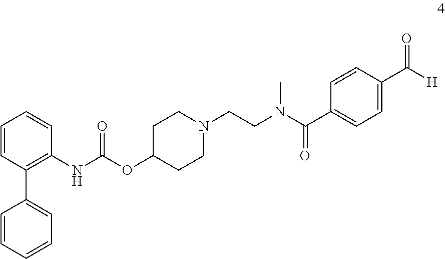 [SMILES: CN(CCN1CCC(OC(=O)Nc2ccccc2-c2ccccc2)CC1)C(=O)c1ccc(C=O)cc1]
d) contacting Compound 4 with piperidine-4-carboxamide under conditions to provide revefenacin: 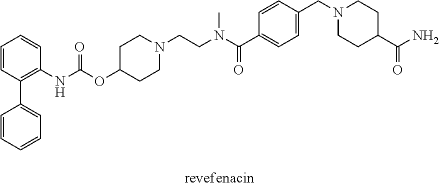 [SMILES: CN(CCN1CCC(OC(=O)Nc2ccccc2-c2ccccc2)CC1)C(=O)c1ccc(CN2CCC(C(N)=O)CC2)cc1]
and optionally:
e) crystallizing the revefenacin under conditions sufficient to provide the crystalline revefenacin Form I or crystalline revefenacin Form II.

14. The process of claim 13, wherein the quantity of Compound 1 added is between 67-72% by weight the quantity of Compound A.

15. A compound which is benzyl (E)-(2-(2-(2,4-dinitrophenyl)hydrazineylidene)ethyl)(methyl)carbamate (Compound 10):

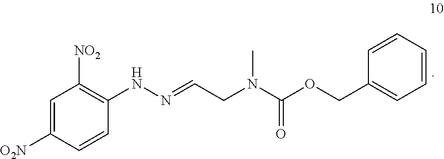 [SMILES: CN(C/C=N/Nc1ccc([N+](=O)[O-])cc1[N+](=O)[O-])C(=O)OCc1ccccc1]

16. A process for determining the quantity of Compound 1 in a composition comprising reacting an aliquot of a composition comprising Compound 1 with 2,4-dinitrophenylhydrazine under conditions sufficient to provide benzyl (E)-(2-(2-(2,4-dinitrophenyl)hydrazineylidene)ethyl)(methyl)carbamate (Compound 10): 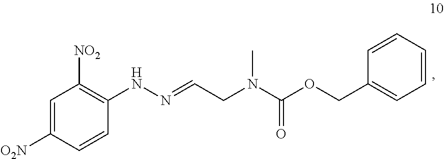 [SMILES: CN(C/C=N/Nc1ccc([N+](=O)[O-])cc1[N+](=O)[O-])C(=O)OCc1ccccc1]
and determining the quantity of benzyl (E)-(2-(2-(2,4-dinitrophenyl)hydrazineylidene)ethyl)(methyl)carbamate by HPLC.

17. A pharmaceutical composition comprising crystalline revefenacin Form III and/or Form IV, wherein the pharmaceutical composition comprises not less than 90.0% revefenacin, and one or more of:
more than 0.05% of Compound 4A: 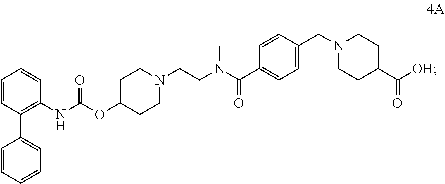 [SMILES: CN(CCN1CCC(OC(=O)Nc2ccccc2-c2ccccc2)CC1)C(=O)c1ccc(CN2CCC(C(=O)O)CC2)cc1]
more than 0.1% of Compound 4B: 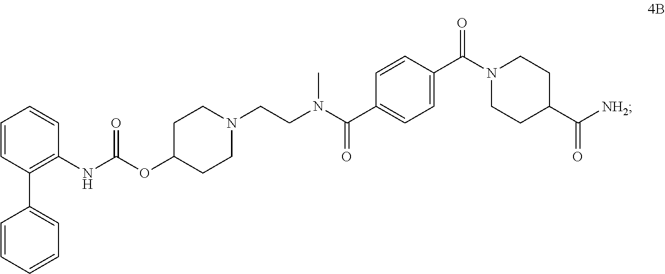 [SMILES: CN(CCN1CCC(OC(=O)Nc2ccccc2-c2ccccc2)CC1)C(=O)c1ccc(C(=O)N2CCC(C(N)=O)CC2)cc1]
more than 0.71% of Compound 4C: 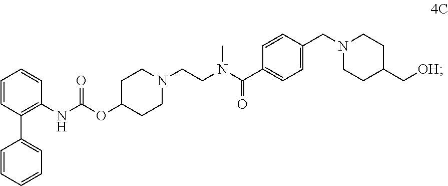 [SMILES: CN(CCN1CCC(OC(=O)Nc2ccccc2-c2ccccc2)CC1)C(=O)c1ccc(CN2CCC(CO)CC2)cc1]
more than 0.26% of Compound 4D: 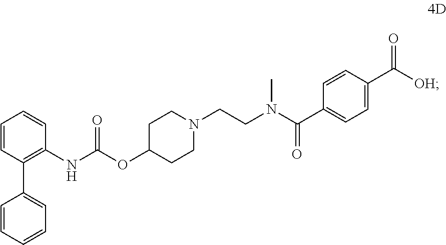 [SMILES: CN(CCN1CCC(OC(=O)Nc2ccccc2-c2ccccc2)CC1)C(=O)c1ccc(C(=O)O)cc1]
more than 0.22% of Compound 4E: 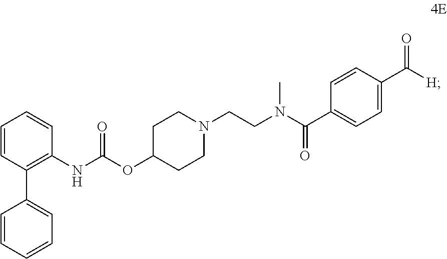
more than 0.23% of a combination of Compound 4F and Compound 4G: 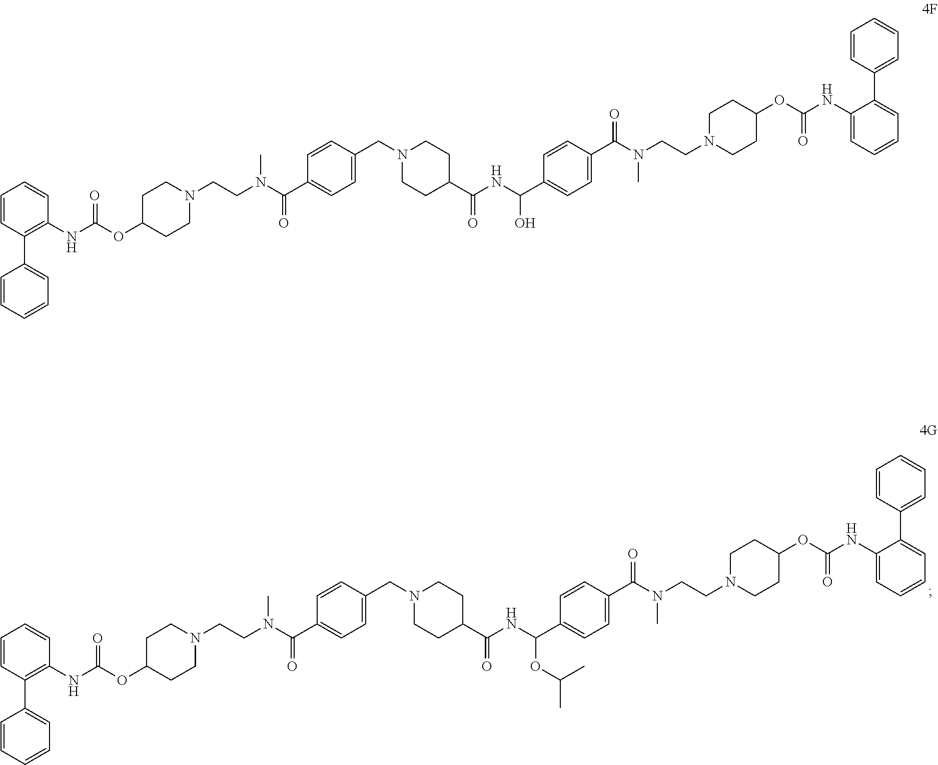 [SMILES: CN(CCN1CCC(OC(=O)Nc2ccccc2-c2ccccc2)CC1)C(=O)c1ccc(CN2CCC(C(=O)NC(O)c3ccc(C(=O)N(C)CCN4CCC(OC(=O)Nc5ccccc5-c5ccccc5)CC4)cc3)CC2)cc1 ; CC(C)OC(NC(=O)C1CCN(Cc2ccc(C(=O)N(C)CCN3CCC(OC(=O)Nc4ccccc4-c4ccccc4)CC3)cc2)CC1)c1ccc(C(=O)N(C)CCN2CCC(OC(=O)Nc3ccccc3-c3ccccc3)CC2)cc1]
more than 0.11% of Compound 4F: 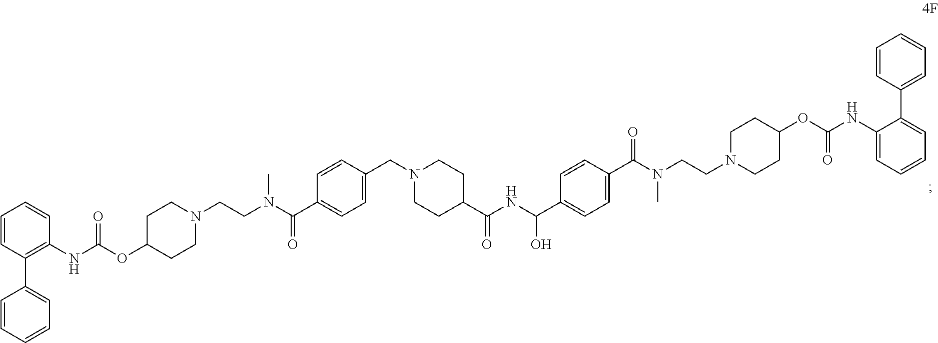 [SMILES: CN(CCN1CCC(OC(=O)Nc2ccccc2-c2ccccc2)CC1)C(=O)c1ccc(CN2CCC(C(=O)NC(O)c3ccc(C(=O)N(C)CCN4CCC(OC(=O)Nc5ccccc5-c5ccccc5)CC4)cc3)CC2)cc1]
more than 0.13% of Compound 4G: 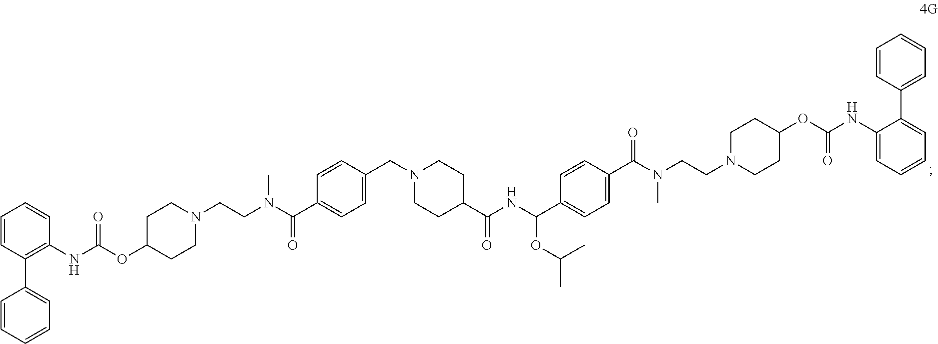 [SMILES: CC(C)OC(NC(=O)C1CCN(Cc2ccc(C(=O)N(C)CCN3CCC(OC(=O)Nc4ccccc4-c4ccccc4)CC3)cc2)CC1)c1ccc(C(=O)N(C)CCN2CCC(OC(=O)Nc3ccccc3-c3ccccc3)CC2)cc1]
or
more than 0.40% of Compound 4H:

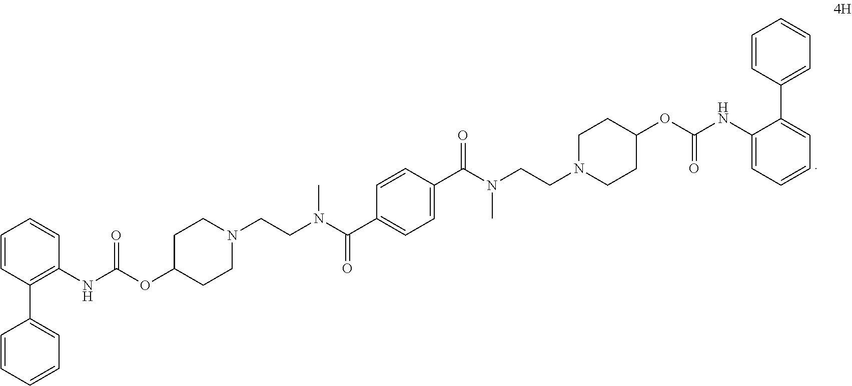 [SMILES: CN(CCN1CCC(OC(=O)Nc2ccccc2-c2ccccc2)CC1)C(=O)c1ccc(C(=O)N(C)CCN2CCC(OC(=O)Nc3ccccc3-c3ccccc3)CC2)cc1]

18. A pharmaceutical composition comprising crystalline revefenacin Form III and/or Form IV, wherein the pharmaceutical composition comprises not less than 90.0% revefenacin, and one or more of:
from 0.05% to 0.40% of Compound 4A: 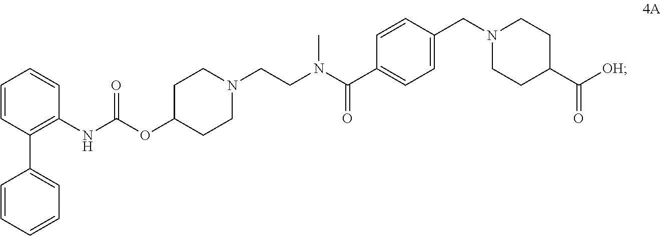 [SMILES: CN(CCN1CCC(OC(=O)Nc2ccccc2-c2ccccc2)CC1)C(=O)c1ccc(CN2CCC(C(=O)O)CC2)cc1]
from 0.1% to 0.40% of Compound 4B: 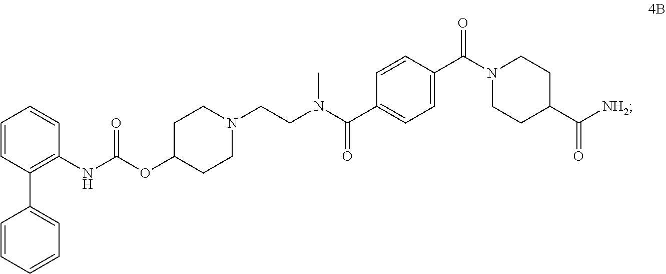 [SMILES: CN(CCN1CCC(OC(=O)Nc2ccccc2-c2ccccc2)CC1)C(=O)c1ccc(C(=O)N2CCC(C(N)=O)CC2)cc1]
from 0.71% to 1.0% of Compound 4C: 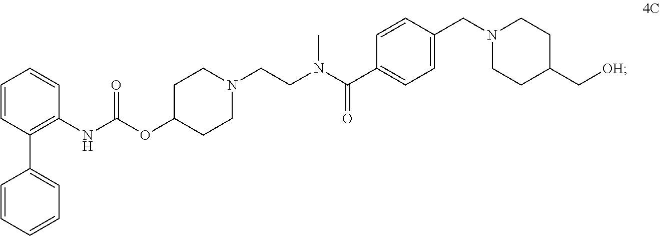 [SMILES: CN(CCN1CCC(OC(=O)Nc2ccccc2-c2ccccc2)CC1)C(=O)c1ccc(CN2CCC(CO)CC2)cc1]
from 0.26% to 0.40% of Compound 4D: 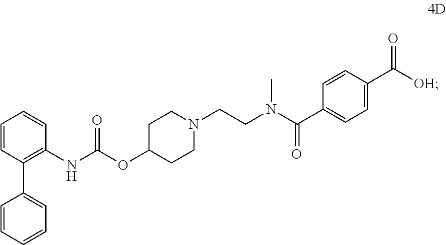 [SMILES: CN(CCN1CCC(OC(=O)Nc2ccccc2-c2ccccc2)CC1)C(=O)c1ccc(C(=O)O)cc1]
from 0.22% to 0.70% of Compound 4E: 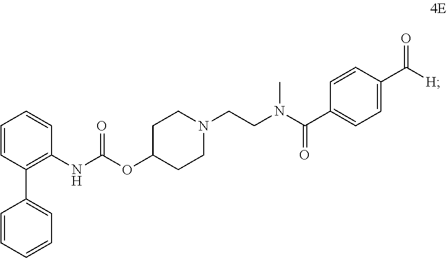
or
from 0.40% to 0.70% of Compound 4H:

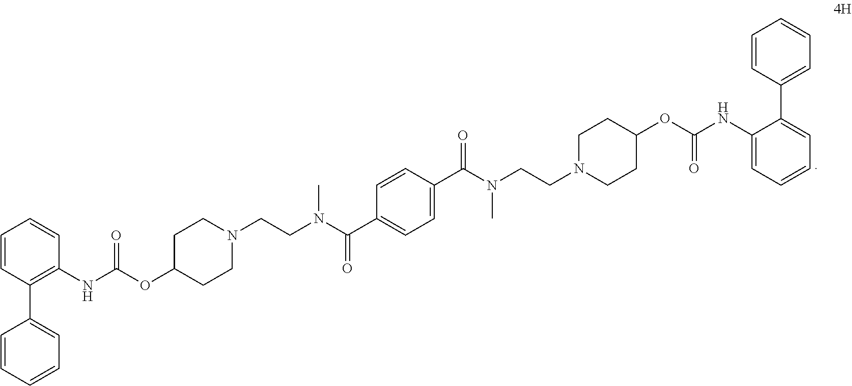 [SMILES: CN(CCN1CCC(OC(=O)Nc2ccccc2-c2ccccc2)CC1)C(=O)c1ccc(C(=O)N(C)CCN2CCC(OC(=O)Nc3ccccc3-c3ccccc3)CC2)cc1]

19. A pharmaceutical composition comprising revefenacin, wherein the revefenacin in the pharmaceutical composition is substantially crystalline revefenacin Form I, substantially crystalline revefenacin Form II, or substantially amorphous revefenacin, wherein the pharmaceutical composition comprises not less than 90.0% revefenacin, and not more than:
0.40% of Compound 4A: 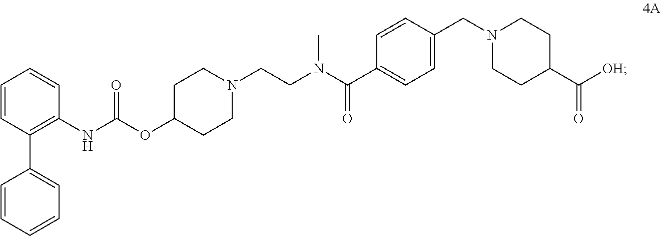 [SMILES: CN(CCN1CCC(OC(=O)Nc2ccccc2-c2ccccc2)CC1)C(=O)c1ccc(CN2CCC(C(=O)O)CC2)cc1]
0.40% of Compound 4B: 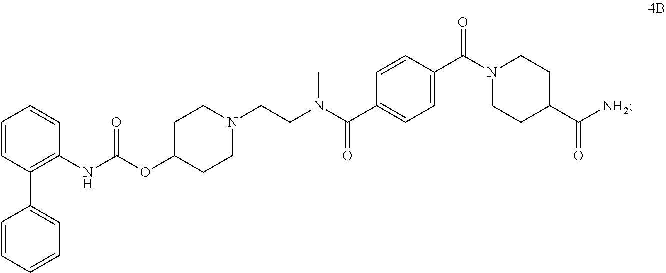 [SMILES: CN(CCN1CCC(OC(=O)Nc2ccccc2-c2ccccc2)CC1)C(=O)c1ccc(C(=O)N2CCC(C(N)=O)CC2)cc1]
1.0% of Compound 4C: 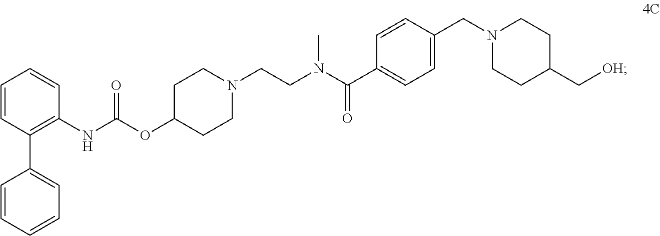 [SMILES: CN(CCN1CCC(OC(=O)Nc2ccccc2-c2ccccc2)CC1)C(=O)c1ccc(CN2CCC(CO)CC2)cc1]
0.40% of Compound 4D: 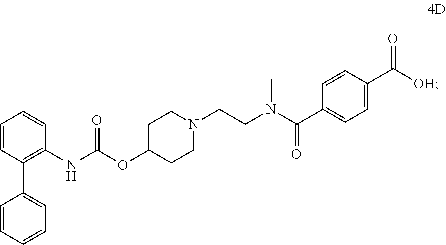 [SMILES: CN(CCN1CCC(OC(=O)Nc2ccccc2-c2ccccc2)CC1)C(=O)c1ccc(C(=O)O)cc1]
0.70% of Compound 4E: 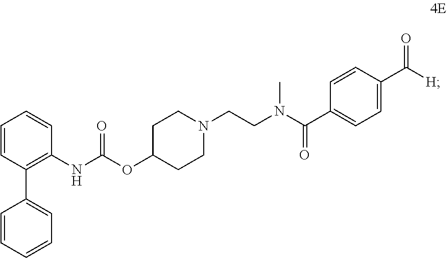
or
0.70% of Compound 4H: 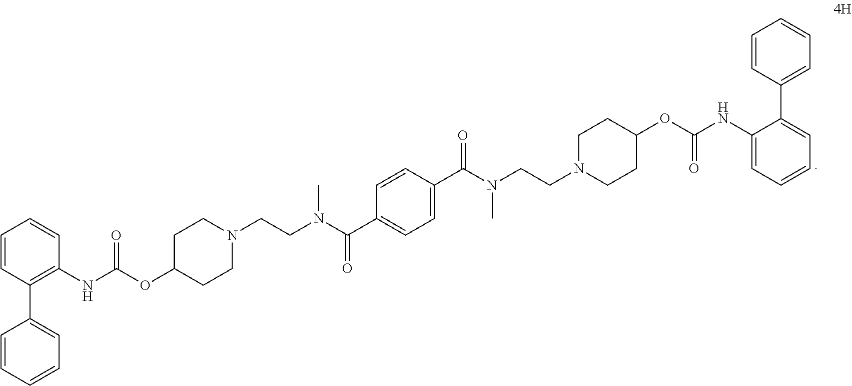 [SMILES: CN(CCN1CCC(OC(=O)Nc2ccccc2-c2ccccc2)CC1)C(=O)c1ccc(C(=O)N(C)CCN2CCC(OC(=O)Nc3ccccc3-c3ccccc3)CC2)cc1]
